(19) United States
(12) Patent Application Publication
(10) Pub. No.: US 2024/0325395 A1
(43) Pub. Date: Oct. 3, 2024

(54) REAGENT AND METHOD FOR TREATING SKIN DISEASES OR CONDITIONS ASSOCIATED WITH ANTI-TUMOR AGENT

(71) Applicant: ONQUALITY PHARMACEUTICALS CHINA LTD., Shanghai (CN)

(72) Inventors: Wenxi LI, Shanghai (CN); Yang YAO, Shanghai (CN); Linan YANG, Shanghai (CN); Shiyi ZHANG, Shanghai (CN)

(73) Assignee: ONQUALITY PHARMACEUTICALS CHINA LTD., Shanghai (CN)

(21) Appl. No.: 18/259,532

(22) PCT Filed: Dec. 28, 2021
(86) PCT No.: PCT/CN2021/141973

§ 371 Date: Jun. 27, 2023

(30) Foreign Application Priority Data
Dec. 29, 2020 (CN) 202011602295.1
Jun. 7, 2021 (CN) 202110630457.0

(51) Int. Cl. A61K 31/519; A61P 17/00; A61P 39/00

(52) U.S. Cl. CPC A61K 31/519; A61P 17/00; A61P 39/00

(57) ABSTRACT

A method containing a compound as shown in formula 1, or a pharmaceutically acceptable salt, prodrug, isotopic variant or solvate thereof in preparation of a medicament:

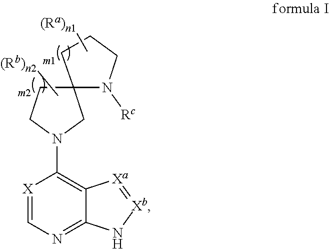

The medicament is suitable for preventing, relieving, and/or treating skin diseases or disorders associated with anti-tumor agents in a subject. Also disclosed is a method for treating skin diseases or disorders associated with anti-tumor agents by the compound.

### TECHNICAL FIELD

The present application relates to the field of biomedicine, and particularly to a reagent and method of preventing, relieving and/or treating a skin disease or disorder associated with an antitumor agent.

### BACKGROUND

Chemotherapy, radiotherapy and surgery are the most commonly used methods for the treatment of tumors in clinical practice. The selectivity of chemotherapy is not high because it not only kills tumor cells, but also causes damages to normal cells with great side effects. Compared with traditional anti-tumor regimens, targeted therapy targets specific targets on tumor cells (such as a specific genetic mutation), and immunotherapy attacks tumor cells by use of the immune system of the body. However, because it cannot differentiate tumor cells from normal cells completely, or it may cause abnormal activation of the immune system, so side effects would still be produced, such as bone marrow suppression, digestive system toxicity, dermal toxicity, renal toxicity or hepatotoxicity, thereby adversely affecting the therapeutic effects, and serious adverse events may be life-threatening, and the survival time of patients may be shortened.

At present, no effective therapeutic regimen has been available yet for controlling the side effects associated with the antitumor agent. Therefore, there is an urgent need for therapeutic regimens that can successfully control these side effects.

### SUMMARY OF THE INVENTION

The present application provides a method of preventing, relieving and/or treating skin side effects (e.g., a skin disease or disorder) associated with an antitumor agent in a subject, which comprises administering a compound as shown in Formula I or a pharmaceutically acceptable salt, prodrug, isotopic variant or solvate thereof. The method of the present application is capable of effectively preventing, relieving and/or treating a skin disease or disorder (e.g., rash) associated with an antitumor agent in a subject, thus greatly improving the life quality of the cancer patient.

In one aspect, the present application provides a use of a compound as shown in Formula I or a pharmaceutically acceptable salt, prodrug, isotopic variant (e.g., completely or partially deuterated form) or solvate thereof in preparing a medicament, the medicament is used for preventing, relieving and/or treating a skin disease or disorder associated with an antitumor agent in a subject:

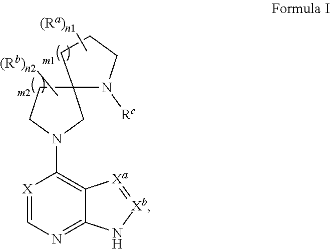

wherein,
R^{a} is the same or different and each is: (1) C₁₋₆ alkyl, or (2) halogen atom;
n1 is an integer selected from 0 to 4;
R^{b} is the same or different and each is: (1) C₁₋₆ alkyl, or (2) halogen atom;
n2 is an integer selected from 0 to 4;
m1 is an integer selected from 0 to 3;
m2 is an integer selected from 1 to 4;
X^{a}═X^{b} is (1) CH═CH, (2) N═CH, or (3) CH═N;
X is (1) nitrogen atom, or (2) C—R^{d} wherein R^{d} is hydrogen atom or halogen atom;
R^{c} is a group selected from the following (1) to (6): (1) hydrogen atom, (2) C₁₋₆ alkyl optionally substituted by the same or different 1 to 5 substituents selected from the following Group A, (3) —C(═O)—R^{c1}, (4) —C(═O)—O—R^{c2}, (5) —C(═O)NR^{c3}R^{c4}, wherein R^{c1}, R^{c2}, R^{c3} and R^{c4} are the same or different and each is; hydrogen atom, or (ii) C₁₋₆ alkyl optionally substituted by the same or different 1 to 5 substituents selected from the following Group A, or (6) a group with a structure of

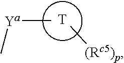

wherein,
Ya is a group selected from the following, (i) to (iii): (i) C₁₋₆ alkylene, (ii) —C(═O)—, or (iii) —C(═O)—O—,
Ring T is (i) C₆₋₁₀ aryl, (ii) C₃₋₁₀ cycloalkyl, or (iii) saturated monoheterocyclyl containing 1 to 4 heteroatoms selected from nitrogen atom, oxygen atom or sulfur atom as well as carbon atoms, and the number of the constituent ring atoms is 3 to 7,
R^{c5} is the same or different and each is: (i) cyanogroup, or (ii) nitro, p is an integer selected from 0 to 4;
Group A is the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

In some embodiments, X^{a} is carbon atom and X^{b} is carbon atom. In some embodiments, X^{a} is nitrogen atom and X^{b} is carbon atom. In some embodiments, X^{a} is carbon atom and X^{b} is nitrogen atom.

In some embodiments, in Formula I: n1 is an integer selected from 0 to 2; n2 is an integer selected from 0 to 2: m1 is an integer selected from 0 to 3; m2 is an integer selected from 1 to 3; X is (1) nitrogen atom, or (2) C—R^{d}, wherein R^{d} is halogen atom; R^{c} is a group selected from the following (1) to (6): (1) hydrogen atom, (2) C₁₋₆ alkyl substituted by one substituent selected from the following Group A, (3) —C(═O)—R^{c1}, (4) —C(═O)—O—R^{c2}, (5) —C(═O)—NR^{c3}R^{c4}, wherein R^{c2} is C₁₋₆ alkyl optionally substituted by one substituent selected from the following Group A, R² is C₁₋₆ alkyl, R^{c3} is C₁₋₆ alkyl optionally substituted by one substituent selected from the following Group A, R^{c4} is (i) hydrogen atom, or (ii) C₁₋₆ alkyl, or (6) a group with a structure of

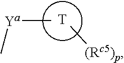

wherein, Y^{a} is a group selected from the following (i) to (iii): (i) C₁₋₆ alkylene, (ii) —C(═O)—, or (iii) —C(═O)—O—, ring T is (i) phenyl, (ii) C₃₋₆ cycloalkyl, or (iii) pyrrolidinyl, R^{c5} is (i) cyanogroup, or (ii) nitro, p is an integer selected from 0 or 1; Group A is the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

In some embodiments, m1 is an integer selected from 0 or 1, m2 is an integer selected from 1 or 2.

In some embodiments, m1 is 1, m2 is 2, the compound is a compound as shown in Formula II:

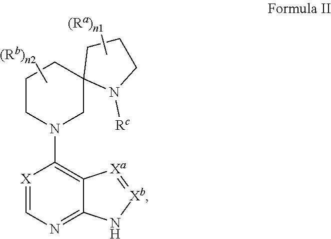

wherein, R^{a}, R^{b}, R^{c}, X, X^{a}, X^{b}, n1 and n2 are as defined above.

In some embodiments, m1 is 0, m2 is 2, the compound is a compound of Formula III:

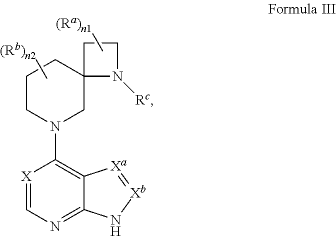

wherein R^{a}, R^{b}, R^{c}, X, X^{a}, X^{b}, n1 and n2 are as defined above.

In some embodiments, m1 is 0, m2 is 1, the compound is a compound of Formula IV:

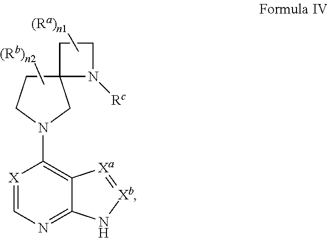

wherein R^{a} is the same or different and each: (1) C₁₋₆ alkyl, or (2) halogen atom:
n1 is an integer selected from 0 to 4;
R^{b} is the same or different and each: (1) C₁₋₆ alkyl, or (2) halogen atom;
n2 is an integer selected from 0 to 4;
X^{a}═X^{b} is (1) CH═CH, (2) N═CH, or (3) CH═N;
X is (1) nitrogen atom, or (2) C—R^{d}, wherein R^{d} is hydrogen atom or halogen atom;
R^{c} is a group selected from the following (1) to (6): (1) hydrogen atom, (2) C₁₋₆ alkyl optionally substituted by the same or different 1 to 5 substituents selected from the following Group A, (3) —C(═O)—R^{c1}, (4) —C(═O)—O—R^{c2}, (5) —C(═O)—NR^{c3}R^{c4}, wherein R^{c1}, R^{c2}, R^{c3} and R^{c4} are the same or different and each is: hydrogen atom, or (ii) C₁₋₆ alkyl optionally substituted by the same or different 1 to 5 substituents selected from the following Group A, or (6) a group of a structure of

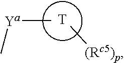

wherein,
Y^{a} is a group selected from the following (i) to (iii): (i) c₁₋₆ alkylene, (ii) —C(═O)—, or (iii) —C(═O)—O—,
Ring T is (i) C₆₋₁₀ aryl, (ii) C₃₋₁₀ cycloalkyl, or (iii) saturated monoheterocyclyl containing 1 to 4 heteroatoms selected from nitrogen atom, oxygen atom or sulfur atom as well as carbon atoms- and the number of the constituent ring atoms is 3 to 7,
R^{c5} is the same or different and each: (i) cyanogroup, or (ii) nitro, p is an integer selected from 0 to 4;
Group A is the group consisting of: (a) hydroxyl, (b) C₁₋₆ alkoxy (c) cyanogroup, (d) C₁₋₆ alkoxycarbonyl, (e) C₁₋₆ alkylcarbonyloxy, and (f) C₂₋₆ alkenyloxy.

In some embodiments, X^{a} is carbon atom and X^{b} is carbon atom. In some embodiments. X^{a} is nitrogen atom and X^{b} is carbon atom. In some embodiments, X^{a} is carbon atom and X^{b} is nitrogen atom.

In some embodiments, in Formula I: n1 is an integer selected from 0 to 2; n2 is an integer selected from 0 to 2; m1 is an integer selected from 0 to 3: m2 is an integer selected from 1 to 3; X is (1) nitrogen atom, or (2) C—R^{d}, wherein R^{d} is halogen atom; R^{c} is a group selected from the following (1) to (6): (1)hydrogen atom, (2) C₁₋₆ alkyl substituted by one substituent selected from the following Group A, (3) —C(═O)—R^{c1}, (4) —C(═O)—O—R^{c2}, (5) —C(═O)NR^{c3}R^{c4}, wherein R^{c1} is C₁₋₆ alkyl optionally substituted by one substituent selected from the following Group A, R^{c2} is C₁₋₆ alkyl, R^{c4} is C₁₋₆ alkyl optionally substituted by one substituent selected from the following Group A, R^{c4} is (i) hydrogen atom, or (ii) C₁₋₆ alkyl, or (6) a group with a structure of

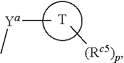

wherein, Y^{a} is a group selected from the (i) to (iii): (i) C₁₋₆ alkylene, (ii) —C(═O)—, or (iii) —C(═O)—O—, Ring T is (i) phenyl, (ii) C₃₋₆ cycloalkyl, or (iii) pyrrolidinyl, R^{c5} is (i) cyanogroup, or (ii) nitro, p is an integer selected from 0 or 1, Group A is the group consisting of: (a) hydroxyl, (b) C₁₋₆ alkoxy, (c) cyanogroup, (d) C₁₋₆ alkoxycarbonyl, (e) C₁₋₆ alkyl carbonyloxy, and (f) C₂₋₆ alkenyloxy.

In some embodiments, the m1 and m2 are selected from the following group: (1) m1 is 0, m2 is 3, (2) m1 is 2, m2 is 1, (3) m1 is 2, and (4) m1 is 3, m2 is 2.

In some embodiments, X^{a}═X^{b} is CH═CH, X is nitrogen atom. In some embodiments, X^{a} is carbon atoms, X^{b} is carbon atoms, X is nitrogen atom.

In some embodiments, n1 is 0, and n2 is 0.

In some embodiments, n1 is 1, and n2 is 0.

In some embodiments, n1 is 0, and n2 is 1.

In some embodiments, n1 is 2, and n2 is 0.

In some embodiments, n1 is 0, and n2 is 2.

In some embodiments, R^{a} is methyl or fluorine atom.

In some embodiments, R^{c} is —C(═O)—R^{c1}.

In some embodiments, R^{c} is C₁₋₆ alkyl substituted by one hydroxyl or cyanogroup.

In some embodiments, R^{c} is —C(═O)—R^{c3}R^{c4}.

In some embodiments, R^{c3} is C₁₋₆ alkyl substituted by one cyanogroup, R^{c4} is hydrogen.

In some embodiments, the compound as shown in Formula I is selected from the following group:

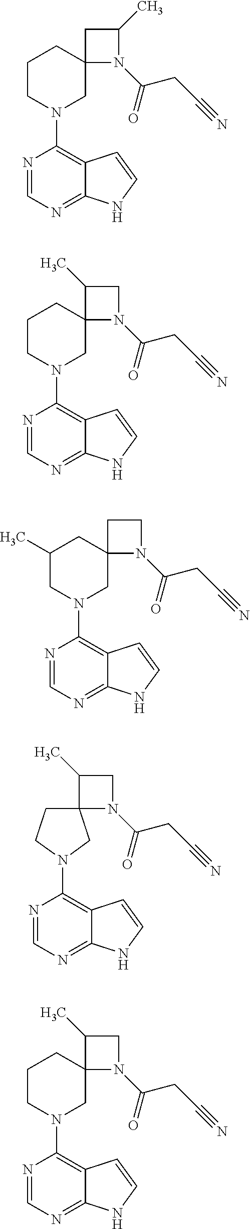 [SMILES: CC1CC2(CCCN(c3ncnc4[nH]ccc34)C2)N1C(=O)CC#N ; CC1CN(C(=O)CC#N)C12CCCN(c1ncnc3[nH]ccc13)C2 ; CC1CN(c2ncnc3[nH]ccc23)CC2(CCN2C(=O)CC#N)C1 ; CC1CN(C(=O)CC#N)C12CCN(c1ncnc3[nH]ccc13)C2 ; CC1CN(C(=O)CC#N)C12CCCN(c1ncnc3[nH]ccc13)C2]

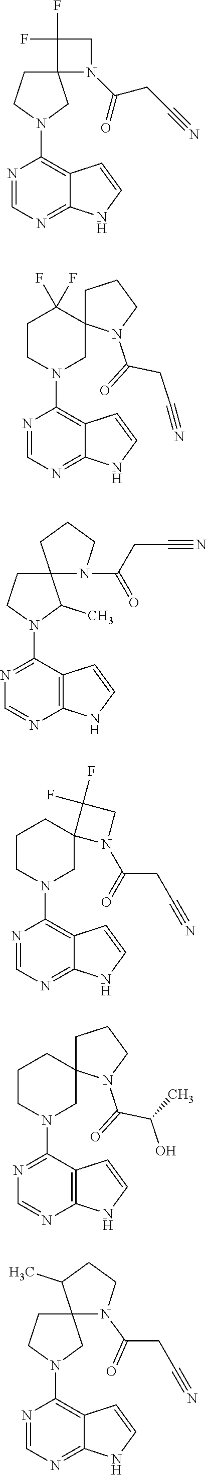 [SMILES: N#CCC(=O)N1CC(F)(F)C12CCN(c1ncnc3[nH]ccc13)C2 ; N#CCC(=O)N1CCCC12CN(c1ncnc3[nH]ccc13)CCC2(F)F ; CC1N(c2ncnc3[nH]ccc23)CCC12CCCN2C(=O)CC#N ; N#CCC(=O)N1CC(F)(F)C12CCCN(c1ncnc3[nH]ccc13)C2 ; C[C@H](O)C(=O)N1CCCC12CCCN(c1ncnc3[nH]ccc13)C2 ; CC1CCN(C(=O)CC#N)C12CCN(c1ncnc3[nH]ccc13)C2]

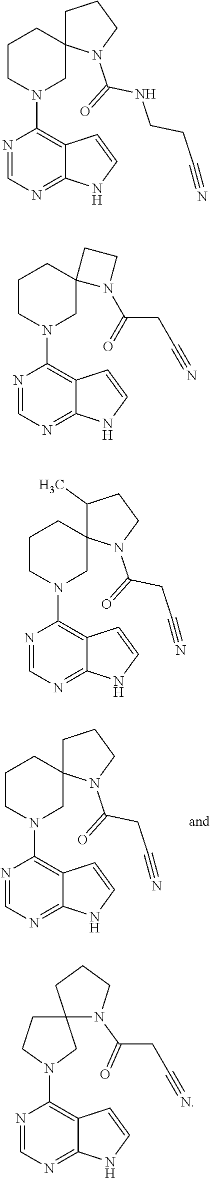 [SMILES: N#CCCNC(=O)N1CCCC12CCCN(c1ncnc3[nH]ccc13)C2 ; N#CCC(=O)N1CCC12CCCN(c1ncnc3[nH]ccc13)C2 ; CC1CCN(C(=O)CC#N)C12CCCN(c1ncnc3[nH]ccc13)C2 ; N#CCC(=O)N1CCCC12CCCN(c1ncnc3[nH]ccc13)C2 ; N#CCC(=O)N1CCCC12CCN(c1ncnc3[nH]ccc13)C2]

In some embodiments, the compound as shown in Formula I is selected from the following group:

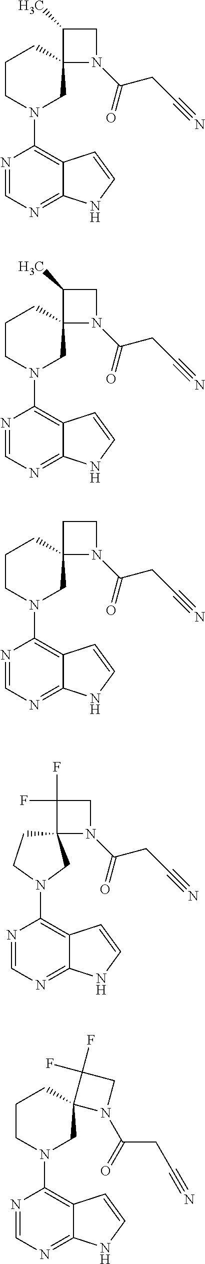 [SMILES: C[C@@H]1CN(C(=O)CC#N)[C@]12CCCN(c1ncnc3[nH]ccc13)C2 ; C[C@H]1CN(C(=O)CC#N)[C@]12CCCN(c1ncnc3[nH]ccc13)C2 ; N#CCC(=O)N1CC[C@@]12CCCN(c1ncnc3[nH]ccc13)C2 ; N#CCC(=O)N1CC(F)(F)[C@@]12CCN(c1ncnc3[nH]ccc13)C2 ; N#CCC(=O)N1CC(F)(F)[C@@]12CCCN(c1ncnc3[nH]ccc13)C2]

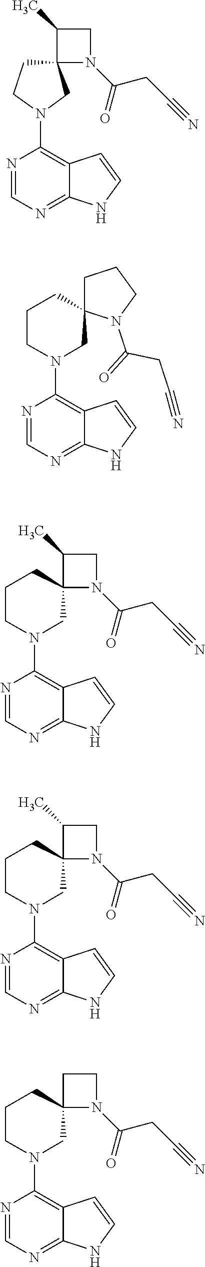 [SMILES: C[C@H]1CN(C(=O)CC#N)[C@]12CCN(c1ncnc3[nH]ccc13)C2 ; N#CCC(=O)N1CCC[C@@]12CCCN(c1ncnc3[nH]ccc13)C2 ; C[C@H]1CN(C(=O)CC#N)[C@@]12CCCN(c1ncnc3[nH]ccc13)C2 ; C[C@@H]1CN(C(=O)CC#N)[C@@]12CCCN(c1ncnc3[nH]ccc13)C2 ; N#CCC(=O)N1CC[C@]12CCCN(c1ncnc3[nH]ccc13)C2]

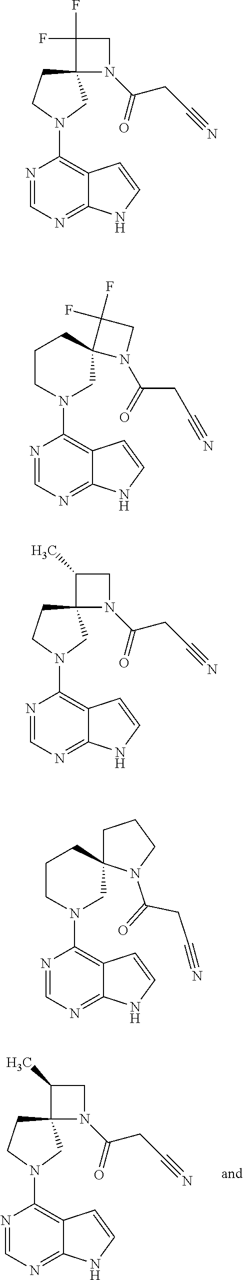 [SMILES: N#CCC(=O)N1CC(F)(F)[C@]12CCN(c1ncnc3[nH]ccc13)C2 ; N#CCC(=O)N1CC(F)(F)[C@]12CCCN(c1ncnc3[nH]ccc13)C2 ; C[C@@H]1CN(C(=O)CC#N)[C@@]12CCN(c1ncnc3[nH]ccc13)C2 ; N#CCC(=O)N1CCC[C@]12CCCN(c1ncnc3[nH]ccc13)C2 ; C[C@H]1CN(C(=O)CC#N)[C@@]12CCN(c1ncnc3[nH]ccc13)C2]

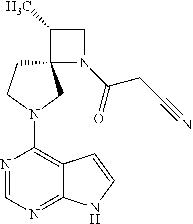 [SMILES: C[C@@H]1CN(C(=O)CC#N)[C@]12CCN(c1ncnc3[nH]ccc13)C2]

In some, embodiments, the compound as shown in Formula I is

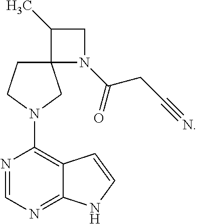 [SMILES: CC1CN(C(=O)CC#N)C12CCN(c1ncnc3[nH]ccc13)C2]

In some embodiments, the antitumor agent comprises a small molecular compound, a small molecular conjugate, protein and/or polynucleotide.

In some embodiments, the antitumor agent comprises targeted therapeutic agent and/or immunotherapeutic agent.

In some embodiments, the antitumor agent is targeted therapeutic agent.

In some embodiments, the targeted therapeutic agent comprises a small molecular compound and/or an antibody or fragment of antigen binding thereof.

In some embodiments, the antibody comprises monoclonal antibody, polyclonal antibody, chimeric antibody, humanized antibody, fully human antibody and/or antibody-drug conjugate.

In some embodiments, the fragment of antigen binding comprises Fab, Fab′, F(ab)₂, Fv fragment, F(ab′)₂, scFv, di-scFv and/or dAb.

In some embodiments, the targeted therapeutic agent targets molecules within tumor cells, on cell surface and/or in tumor microenvironment.

In some embodiments, the targeted therapeutic agent targets protein and/or nucleic acid molecules of tumor cells.

In some embodiments, the targeted therapeutic agent targets tumor antigen.

In some embodiments, the targeted therapeutic agent targets EGFR, ALK, MEK, VEGFR, FGFR, PDGFR, ABL, BTK, KIT, AKT, mTOR, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, SRC, CD20, PD-L1 and/or BRCA1/2, or mutants thereof.

In some embodiments, the targeted therapeutic agent comprises a hormonotherapy, a signal transduction inhibitor, a gene expression modulator, a cell apoptosis inducer, an angiogenesis inhibitor and/or a toxin delivery molecule.

In some embodiments, the targeted therapeutic agent is a tyrosine kinase inhibitor.

In some embodiments, the targeted therapeutic agent is selected from the following group: EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor and/or SRC inhibitor, as well as a combination thereof.

In some embodiments, the targeted therapeutic agent is an EGFR inhibitor.

In some embodiments, the targeted therapeutic agent is a VEGFR inhibitor.

In some embodiments, the targeted therapeutic agent is a FGFR inhibitor.

In some embodiments, the targeted therapeutic agent is an ALK inhibitor.

In some embodiments, the targeted therapeutic agent is an mTOR inhibitor.

In some embodiments, the targeted therapeutic agent is a BTK inhibitor.

In some embodiments, the targeted therapeutic agent is a MEK inhibitor.

In some embodiments, the targeted therapeutic agent is a PI3K inhibitor.

In some embodiments, the targeted therapeutic agent is an EGFR/cMET double-target inhibitor.

In some embodiments, the antitumor agent is an immunotherapeutic agent.

In some embodiments, the immunotherapeutic agent can change the immune response in a subject.

In some embodiments, the immunotherapeutic agent can enhance the immune response in a subject.

In some embodiments, the immunotherapeutic agent is an immune checkpoint inhibitor, a modified immune cell and/or a vaccine.

In some embodiments, the immunotherapeutic agent is an antibody.

In some embodiments, the immunotherapeutic agent is selected from the following group: PD-1 inhibitor, PD-L1 inhibitor and/or CTLA-4 inhibitor, as well as a combination thereof.

In some embodiments, the antitumor agent is selected from the following group: afatinib, dacomitinib, osimertinib, EAI045, gefitinib, almonertinib, pyrotinib, brigatinib, neratinib, olmutinib, bosutinib, icotinib, vandetanib, lapatinib, alflutinib, BPI-7711, mobocertinib, dovitinib, zorifertinib, varlitinib, orelabrutinib, tirabrutinib, zanubrutinib, acalabrutinib, ibrutinib, dasatinib, pirtobrutinib, tolebrutinib, rilzabrutinib, fenebrutinib, evobrutinib, selumetinib, binimetinib, cobimetinib, trametinib, regorafenib, GSK-1120212, alpelisib, duvelisib, copanlisib, idelalisib, nortriptyline, inavolisib, dactolisib, apitolisib, parsaclisib, buparlisib, rigosertib, enzastaurin, paxalisib, leniolisib, ipatasertib, zotarolimus, sirolimus, everolimus, temsirolimus, sorafenib, apatinib, lenvatinib, sunitinib, cabozantinib, axitinib, nintedanib, brivanib, vatalanib, fruquintinib, dabrafenib, vemurafenib, encorafenib, pazopanib, crizotinib, panobinostat, erlotinib, rituximab, panitumumab, cetuximab, ticilimumab, erfonrilimab, BA-3071, MEDI-5752, defactinib, zalifrelimab, cadonilimab, BCD-217, ipilimumab, tremelimumab, quavonlimab, atezolizumab, durvalumab, camrelizumab, tislelizumab, sintilimab, toripalimab, pembrolizumab, nivolumab, amivantamab, MCLA-129, EMB-01, LY3164530, Roche Glycart anti-EGFR/cMet, Genentech Anti-met/EGFR, Samsung Anti-EGFR/cMet, Merck serono Anti-cMet/EGFR, GB263 and lazertinib, as well as a combination thereof.

In some embodiments, the skin disease or disorder comprises cutaneous disease and/or subcutaneous tissue disease caused by the antitumor agent.

In some embodiments, the skin disease or disorder comprises skin disease or disorder associated with a combination of two or more of the antitumor agents.

In some embodiments, the skin disease or disorder comprises skin diseases or disorders associated with administration of an antitumor agent in combination with one or more other therapies. In some embodiments, the other therapies comprise surgery, radiotherapy and/or chemotherapy. In some embodiments, the skin disease or disorder comprises cutaneous or subcutaneous tissue adverse events caused by the antitumor agent.

In some embodiments, the skin disease or disorder develops or deteriorates after administration of the antitumor agent.

In some embodiments, the skin disease or disorder develops or deteriorates about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 1 day, about 2 days, about 4 days, about 7 days, about 2 weeks, about 3 weeks, about 1 month, about 2 months or later after administration of the antitumor agent.

In some embodiments, the severity grading of the skin disease or disorder increases after administration of the antitumor agent.

In some embodiments, the subject does not suffer from the skin disease or disorder before administration of the antitumor agent.

In some embodiments, the skin disease or disorder comprises alopecia, body odor, dermatitis bullosa, xeroderma, eczema, erythema multiforme, erythroderma, lipoatrophy, hair discolor, abnormal hair texture, hirsutism, hyperhidrosis, hyperkeratosis, hypertrichosis, hypohidrosis, lipohypertrophy, nail changes, nail discolor, nail loss, nail ridge, cutaneous pain, hand-foot syndrome, photosensitivity, pruritus, purpura, acneiform rash, maculopapule, scalp pain, adermotrophia, skin hyperpigmentation, skin hypopigmentation, calluses, skin ulcer, Stevens-Johnson syndrome, subcutaneous emphysema, capillary telangiectasia, toxic epidermal necrolysis, urticaria.

In some embodiments, the skin disease or disorder is rash.

In some embodiments, the severity grading of the skin disease or disorder is Grade 1 or above, Grade 2 or above, Grade 3 or above, Grade 4 or above, or Grade 5, as evaluated in accordance with NCI-CTCAE.

In some embodiments, the subject comprises a cancer patient.

In some embodiments, the site of the skin disease or disorder is different from the site of cancer.

In some embodiments, the medicament does not substantially affect the therapeutic effect of the antitumor agent.

In some embodiments, the antitumor agent is administered in combination with one or more other therapies.

In some embodiments, the medicament is prepared for local administration.

In some embodiments, the medicament is prepared for transdermal administration.

In some embodiments, the medicament is prepared as cream, lotion, gel, ointment, oleamen, spray, liposome preparation, liniment and/or aerosol.

In some embodiments, the administration site of the medicament is different from the administration site of the antitumor agent.

In some embodiments, the administration site of the medicament is not the occurrence site of cancer or potential metastatic site of cancer.

In some embodiments, the medicament is administrated in a manner different from that of the antitumor agent.

In some embodiments, the concentration of the compound as shown in Formula I or a pharmaceutically acceptable salt, prodrug, isotopic variant or solvate thereof in the medicament is about 0.0001% to about 50%.

In some embodiments, the concentration of the compound as shown in Formula I or a pharmaceutically acceptable salt, prodrug, isotopic variant or solvate thereof in the medicament is about 0.010% to about 5.0%.

In another aspect, the present application provides a method of preventing, relieving and/or treating a skin disease or disorder associated with an antitumor agent, comprising administering to a subject in need thereof the compound as shown in Formula I or a pharmaceutically acceptable salt, prodrug, isotopic variant or solvate thereof of the present application.

In some embodiments, the compound as shown in Formula I or a pharmaceutically acceptable salt, prodrug, isotopic variant or solvate thereof is locally administered.

In some embodiments, the compound as shown in Formula I or a pharmaceutically acceptable salt, prodrug, isotopic variant or solvate thereof is transdermally administered.

In some embodiments, the compound as shown in Formula I or a pharmaceutically acceptable salt, prodrug, isotopic variant or solvate thereof is prepared as cream, lotion, gel, ointment, oleamen, spray, liposome preparation, liniment and/or aerosol.

In some embodiments, the subject comprises a cancer patient.

In some embodiments, the cancer patient has been, is being, and/or will be administered with the antitumor agent.

In some embodiments, the concentration of the compound as shown in Formula I or a pharmaceutically acceptable salt, prodrug, isotopic variant or solvate thereof is about 0.0001% to about 50%.

In some embodiments, the concentration of the compound as shown in Formula I or a pharmaceutically acceptable salt, prodrug, isotopic variant or solvate thereof is about 0.01% to about 5.0%.

In some embodiments, the compound as shown in Formula I or a pharmaceutically acceptable salt, prodrug, isotopic variant or solvate thereof is administered before, simultaneously with, or after the administration of the antitumor agent.

In another aspect, the present application provides the compound as shown in Formula I or a pharmaceutically acceptable salt, prodrug, isotopic variant or solvate thereof as described in the present application, which is used for preventing, relieving and/or treating a skin disease or disorder associated with an antitumor agent.

In another aspect, the present application provides a pharmaceutical composition, comprising the compound as shown in Formula I or a pharmaceutically acceptable salt, prodrug, isotopic variant, or solvate thereof as described in the present application, and a pharmaceutically acceptable carrier.

In another aspect, the present application provides a pharmaceutical composition, comprising the compound as shown in Formula I or a pharmaceutically acceptable salt, prodrug, isotopic variant, or solvate thereof as described in the present application, and an antitumor agent.

In another aspect, the present application provides a kit, comprising the compound as shown in Formula I or a pharmaceutically acceptable salt, prodrug, isotopic variant, or solvate thereof as described in the present application, and an antitumor agent.

Other aspects and advantages of the present application will be readily apparent to those skilled in the art from the following detailed description. Only the exemplary embodiments of the present application are shown and described in the following detailed description. As will be appreciated by those skilled in the art, the disclosure of the present application allows persons skilled in the art to modify the disclosed embodiments without departing from the spirit and scope of the invention involved in the present application. Accordingly, the drawings and the description in the specification of the present application are merely exemplary, and not restrictive.

### BRIEF DESCRIPTION OF THE DRAWINGS

Specific features of the invention involved in the present application are set forth in the appended claims. The features and advantages of the invention involved in the present application can be better understood by referring to the exemplary embodiments detailed hereinafter and the accompanying drawings. A brief description of the drawings is as follows:

FIG. 1 shows the photographs of left side, back side, and right side of a rat model where rashes are caused by the antitumor agent of the present application.

FIG. 2 shows the photographs of left side, back side, and right side of typical rats in the control group and the delgocitinib group in examples 1-96 of the present application.

FIG. 3 shows grading results of partial rashes in the control group and the delgocitinib group in examples 1-96 of the present application, wherein, Delgo represents delgocitinib.

FIG. 4 shows grading results of partial rashes in the control group and the delgocitinib group in examples 97-108 of the present application, wherein, Delgo represents delgocitinib.

FIG. 5 shows the photographs of left side, back side, and right side of typical rats in the control group and the delgocitinib group in examples 109-136 of the present application.

FIG. 6 shows grading results of partial rashes in the control group and the delgocitinib group in examples 137-144 of the present application, wherein, Delgo represents delgocitinib.

FIG. 7 shows grading results of partial rashes in the control group and the delgocitinib group in examples 145-157 of the present application, wherein, Delgo represents delgocitinib.

FIG. 8 shows the photographs of left side, back side, and right side of typical rats in other dermatologic medicament group and the delgocitinib group in examples 158-159 of the present application.

FIG. 9 shows grading results of partial rashes in other dermatologic medicament group and the delgocitinib group in examples 160-169 of the present application, wherein, Delgo represents delgocitinib.

### DETAILED DESCRIPTION

The embodiments of the present invention are described below by way of specific examples, and persons skilled in the art can readily appreciate other advantages and effects of the present invention from the disclosure of the present specification.

### Term Definition

In the present application, the term “optionally substituted” generally includes both cases that substitutable positions are substituted and are not substituted (unsubstituted) in the subject group. The term “unsubstituted” refers to such a case that all substitutable positions are substituted by hydrogen atom in the subject group.

In the present application, the term “C₁₋₆ alkyl optionally substituted by the same or different 1 to 5 substituents selected from Group A” includes both cases that substitutable positions of C₁₋₆ alkyl are substituted by the same or different 1 to 5 substituents selected from Group A and not substituted (unsubstituted).

In the present application, the term “halogen atom” includes fluorine atom, chlorine atom, bromine atom or iodine atom. For example, halogen atom may be fluorine atom or chlorine atom.

In the present application, the term “C₁₋₆ alkyl” generally refers to C₁₋₆ straight- or branched-chain saturated hydrocarbon, e.g., methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl. For example, C₁₋₆ alkyl may be methyl, ethyl, propyl, isopropyl, etc.

In the present application, the term “C₂₋₆ alkenyl” generally refers to C₂₋₆ straight- or branched-chain unsaturated hydrocarbon containing one or more double bonds, e.g., vinyl, 1-methylvinyl, 1-propenyl, allyl, methylpropenyl (including 1-methyl-1-propenyl, 2-methyl-1-propenyl, etc.), 1-butenyl, 2-butenyl, 3-butenyl, methylbutenyl (including 1-methyl-1-butenyl, 2-methyl-1-butenyl, 3-methyl-1-butenyl, etc.), pentenyl, methylpentenyl, hexenyl. For example, C₂₋₆ alkenyl may be vinyl, 1-methylvinyl, 1-propenyl, methylpropenyl, etc.

In the present application, the term “C₁₋₆ alkylene” generally refers to a divalent group derived from straight-chain C₁₋₆ alkyl as defined above, e.g., methylene, ethylene, trimethylene, tetramethylene, pentamethylene, hexamethylene. For example, C₁₋₆ alkylene may be methylene, ethylene, etc.

In the present application, the term “C₆₋₁₀ aryl” generally refers to C₆₋₁₀ aromatic hydrocarbon, e.g., phenyl, 1-naphthyl, 2-naphthyl. For example, C₆₋₁₀ aryl may be phenyl.

In the present application, the term “C₃₋₁₀ cycloalkyl” generally refers to C₃₋₁₀ monocyclic saturated hydrocarbon, e.g., cyclopropyl, cyclobutyl, cyclopentyl, cyclohexyl, cycloheptyl, cyclooctyl. For example, C₃₋₁₀ cycloalkyl may be C₃₋₆ cycloalkyl (C₃₋₆ cycloalkyl may include cyclopropyl, cyclobutyl, cyclopentyl, cyclohexyl, etc.).

In the present application, the term “saturated monoheterocyclyl containing 1 to 4 heteroatoms selected from nitrogen atom, oxygen atom or sulfur atom as well as carbon atoms, and the number of the constituent ring atoms is 3 to 7” may generally include oxyranyl, thiolanyl, aziridinyl, azetidinyl, oxetanyl, pyrrolidinyl, pyrrolidino (including 1-pyrrolidinyl), tetrahydrofuranyl, tetrahydrothienyl, oxazolinyl, oxazolidinyl, isoxazolinyl, isoxazolidinyl, thiazolinyl, thiazolidinyl, isothiazolinyl, isothiazolidinyl, imidazolinyl, imidazolidinyl, pyrazolinyl, pyrazolidinyl, piperidinyl, piperidino (including 1-piperidinyl), morpholinyl, morpholino (including 4-morpholinyl), thiomorpholinyl, thiomorpholino (including 4-thiomorpholinyl), piperazinyl, piperazino (including 1-piperazinyl), hexahydro-1,3-oxazinyl, homomorpholine, homopiperazine, etc.

In the present application, the term “C₁₋₆ alkoxy” generally refers to C₁₋₆ straight- or branched-chain alkoxy, particularly methoxy, ethoxy, propoxy, isopropoxy, butoxy, isobutoxy, s-butoxy, t-butoxy, pentyloxy, isopentyloxy, 2-methylbutoxy, neopentyloxy, 1-ethylpropoxy, hexyloxy, etc.

In the present application, the term “C₁₋₆ alkoxycarbonyl” generally refers to a group wherein C₁₋₆ straight- or branched-chain alkoxy binds to carbonyl. For example, C₁₋₆ alkoxycarbonyl may be methoxycarbonyl, ethoxycarbonyl, propoxycarbonyl, isopropoxycarbonyl, butoxycarbonyl, isobutoxycarbonyl, s-butoxycarbonyl, t-butoxycarbonyl, pentyloxycarbonyl, isopentyloxycarbonyl, 2-methylbutoxycarbonyl, neopentyloxycarbonyl, 1-ethylpropoxycarbonyl, hexyloxycarbonyl, 4-methylpentyloxycarbonyl, etc.

In the present application, the term “C₁₋₆ alkylcarbonyloxy” refers to a group wherein a “group wherein C₁₋₆ alkyl binds to carbonyl” binds to an oxygen group. For example, C₁₋₆ alkylcarbonyloxy may be acetyloxy, propionyloxy, butyryloxy, isobutyryloxy, etc.

In the present application, the term “C₂₋₆ alkenyloxy” generally refers to a group wherein “C₂₋₆ alkenyl” binds to oxygen group, e.g., allyloxy, 1-butenyloxy, etc.

In the present application, the term “pharmaceutically acceptable salt” generally refers to any atoxic salts of the compound as shown in Formula I of the present application, including a salt with an inorganic acid, an organic acid, an inorganic base, an organic base, an amino acid, etc. The salt with an inorganic acid includes a salt with hydrochloric acid, nitric acid, sulfuric acid, phosphoric acid, hydrobromic acid, etc. The salt with an organic acid may include a salt with oxalic acid, maleic acid, citric acid, fumaric acid, lactic acid, malic acid, succinic acid, tartaric acid, acetic acid, trifluoroacetic acid, gluconic acid, ascorbic acid, methanesulfonic acid, benzenesulfonic acid, p-toluenesulfonic acid, etc. The salt with an inorganic base may include sodium salt, potassium salt, calcium salt, magnesium salt, ammonium salt, etc. The salt with an organic base may include a salt with methylamine, diethylamine, trimethylamine, triethylamine, ethanolamine, diethanolamine, triethanolamine, ethylenediamine, tris(hydroxymethyl)methylamine, dicyclohexylamine, N, N′-dibenzylethylenediamine, guanidine, pyridine, picoline, choline, cinchonine, meglumine, etc. The salt with an amino acid may include a salt with lysine, arginine, aspartic acid, glutamic acid, etc. According to known methods, each salt may be obtained by reacting a compound as shown in Formula I with an inorganic base, an organic base, an inorganic acid, an organic acid or an amino acid.

In the present application, the term “solvate” generally refers to a material wherein solvent molecules coordinate with a compound as shown in Formula I or a pharmaceutically acceptable salt thereof, and includes hydrate. For example, the solvate may be a pharmaceutically acceptable solvate including 1 hydrate, 1/2 hydrate or 2 hydrate of a compound as shown in Formula I, 1 hydrate of sodium salt of a compound as shown in Formula I, 1 methanolate, 1 ethanolate or 1 acetonitrilate of a compound as shown in Formula I, 2/3 ethanolate of 2 hydrochloride of a compound as shown in Formula I, etc. For example, the solvate may be 1 hydrate of a compound as shown in Formula I. According to known methods, a solvate thereof may be obtained.

In the present application, the compound as shown in Formula I may also exist as various “isomers”. For example, a geometric isomer thereof includes E- and Z-isomers. In case that any asymmetric carbon atoms exist, a stereoisomer based on such carbon atoms includes enantiomers and diastereomers. In case that any chiral axes exist, a stereoisomer based on such axes exists. A tautomer may exist. Therefore, the scope of the present invention encompasses all these isomers and a mixture thereof.

In the present application, the compound as shown in Formula I may be also labeled by isotopes (e.g., ³H, ¹⁴C, ²¹S, etc.).

In the present application, a prodrug of the compound as shown in Formula I may be also a useful drug. The term “prodrug” generally refers to a derivative of the present compound with chemically or metabolically decomposable functional groups, which is administered in vivo, followed by converting into a corresponding parent compound by hydrolysis, solvolysis or physiological decomposition to show original drug efficacies, and includes any composites with noncovalent bonds and salts thereof. The term “parent drug” generally refers to any of the presently described compounds that are useful to treat any of the disorders described herein, or to control or improve the underlying cause or symptoms associated with any physiological or pathological disorders described herein in a host (e.g., human). Prodrugs can be used to achieve any desired effects, including to enhance properties of the parent drug or to improve the pharmaceutic or pharmacokinetic properties of the parent drug. Prodrug strategies exist which provide choices in modulating the conditions for in vivo generation of the parent drug, all of which are deemed included herein. For example, prodrugs can be used to improve the absorption of oral administration, or be used for targeted administration. Modification sited formed by prodrugs include any reactive functional groups of the present compound, including hydroxyl, carboxy, amino, sulphydryl, etc. Nonlimiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others.

In the present application, the term “antibody” generally refers to immunoglobulin reactive to specific protein or peptide or fragment thereof, which generally comprises two identical long chains (heavy chain) and two identical short chains (light chain). Antibodies may be those from any types, including but not limited to IgG, IgA, IgM, IgD and IgE, and those from any subtypes (e.g., IgG1, IgG2, IgG3, and IgG4). An antibody may have a heavy chain constant region selected from e.g., IgG1, IgG2, IgG3, or IgG4. An antibody may also have a light chain selected from e.g., kappa (κ) or lambda (λ). The antibody further comprises artificially-modified antibodies, antibody derivatives, antibody-drug conjugates, antibody analogues or fusion proteins.

In the present application, the term “fragment of antigen binding” generally refers to a certain part of an antibody molecule, which part comprises amino acid residues, and the amino acid residues interact with an antigen and give the antibody specificity and affinity to the antigen. Examples of fragment of antigen binding may include, but not limited to, Fab, Fab′, F(ab)₂, Fv fragment, F(ab′)₂, scFv, di-scFv and/or dAb. In the present application, the term “pharmaceutically acceptable carrier” generally may include various conventional organic or inorganic carriers used as the materials of preparations, e.g., excipient, disintegrant, binder, fluidizer, lubricant for solid preparations, or solvent medium, solubilizing agent, suspending agent, tonicity agent, buffer solution, soothing agent for liquid preparations. Further, an additive including a preservative, an antioxidant, a colorant, a sweetening agent may be used, if needed.

In the present application, the term “antitumor agent” generally refers to any agents capable of inhibiting the occurrence or development of human neoplasms, particularly malignant (cancerous) lesions (e.g., carcinoma, sarcoma, lymphoma or leukemia). The antitumor agent generally achieves the purpose of inhibiting the tumor growth or eliminating the tumor by acting at the cellular and molecular levels through the ways of cell killing, immunoregulation, endocrine regulation, etc. The antitumor agent may include but not limited to, cytotoxic agent, cell growth inhibitor, antiangiogenic agent, cytoreductive agent, chemotherapeutic agent, radiotherapeutic agent, targeted therapeutic agent, biological response modifier, therapeutic antibody, cancer vaccine, cytokines, hormonotherapy, anti-metastasis agent and immunotherapeutic agent. It should be noted that the above classification of antitumor agent does not exclude each other, and one antitumor agent may be classified into one or more categories.

In the present application, the term “targeted therapy” generally refers to cancer therapy using drugs or other substances that interfere with specific molecules (“targets” or “molecular targets”) related to the growth, progression and/or diffusion of cancer cells, while causing little damage to normal cells to achieve anti-tumor effects. The drugs or other substances used in the targeted therapy can be known as “targeted therapeutic agents”. Conversely, drugs used in conventional cytotoxic chemotherapies target all the dividing cells. Targets of targeted therapeutic agent may exist in cancer cells, but do not exist in molecules (e.g., protein or nucleic acid, produced by genetic mutation) of normal cells, or there are more abundant molecules (e.g., protein or nucleic acid) in cancer cells compared with normal cells, particularly those molecules related to the growth or survival of cells. Targets of targeted therapeutic agent may be mutated molecules (e.g., protein or nucleic acid) that drive the progression of cancers. Targets of targeted therapeutic agent may be fusion gene or fusion protein caused by chromosome abnormality. In the present application, the targets may be tumor cell membrane proteins, tumor cell surface receptors, growth factors, hormones or extracellular matrix molecules. The targets also may be protein or nucleic acid molecules within the tumor cells. In the present application, some targeted therapeutic agent may be immunotherapeutic agent, e.g., when the target of the targeted therapeutic agent is involved in the immune response.

In the present application, the term “tyrosine kinase inhibitor” generally refers to any substance or reagent capable of causing the reduction of expression, amount or activity of tyrosine kinase that has been known in the art or will be found in the future, including any substance that causes an inhibition of a biological activity associated with the activity of tyrosine kinase in a subject (including any inhibition of the downstream biological effect caused by the binding of tyrosine kinase with its natural ligand(s)) when the substance is administered with to subject. In some embodiments, tyrosine kinase inhibitor may comprise any reagent capable of blocking the activity of tyrosine kinase or any downstream biological effect thereof during the treatment of cancers. For example, the tyrosine kinase inhibitor can be used for treating tumors. For example, the tyrosine kinase inhibitor can directly inhibit one or more functions of tyrosine kinase. For example, the tyrosine kinase inhibitor can bind to a nucleic acid sequence encoding the tyrosine kinase. For example, the tyrosine kinase inhibitor can reduce the transcriptional level of tyrosine kinase protein. In the present application, no matter in vivo or in vitro, methods for detecting and/or evaluating the inhibition level of tyrosine kinase are common in the art, and such methods can also be used to identify, standardize, screen and/or evaluate the tyrosine kinase inhibitor as described in the present application. For example, the target of the tyrosine kinase inhibitor may be mutated or non-mutated EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, and/or BRCA1/2, or a combination thereof. For example, the tyrosine kinase inhibitor may inhibit one or more targets selected from the following group: EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, and/or BRCA1/2, and mutants thereof. In the present application, the inhibitor (e.g., EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, BCRP inhibitor and/or SRC inhibitor) includes a reagent that reduces the expression of various targets, and/or a reagent that decreases the activities of various targets. In the present application, the inhibitor (e.g., EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor and/or SRC inhibitor) acts directly on proteins of various targets and/or encodes nucleic acids of various target proteins. In the present application, the inhibitor (e.g., EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor and/or SRC inhibitor) comprises a small molecular compound, protein and/or nucleic acid molecules.

In the present application, the term “immunotherapy” generally refers to a method of treating diseases by changing the immunoreaction or immune response of the body, including a method of treating diseases by inducing, enhancing, inhibiting or improving the immunoreaction through acting on the immune system (e.g., immunologic effector cells). The drugs or other substances used to change the immunoreaction or immune response of the body in immunotherapy are known as immunotherapeutic agents. Examples of the immunotherapeutic agents may comprise but not limited to immune cells (generally modified immune cells, e.g., CART cells), antibodies (e.g., monoclonal antibody), vaccines and/or cytokines, etc. The antibodies may be full-length antibodies or fragments of antigen binding thereof, antibody-medicament conjugates (ADC). In the present application, some immunotherapeutic agents may be targeted therapeutic agents, e.g., when the targets of targeted therapeutic agents are involved in the immune response.

In the present application, the term “skin disease or disorder” generally refers to the disease or disorder occurring on skin and accessory organs of skin (including skin, nails and/or mucosa). The definitions and features of the skin disease or disorder may refer to Sections L00-L99 of Chapter XII of The International Statistical Classification of Diseases and Related Health Problems, 10th Revision (ICD-10). In the present application, the skin disease or disorder may be a skin disease or disorder associated with an antitumor agent. In the present application, the skin disease or disorder may be cutaneous tissue or subcutaneous tissue side effects that develop or deteriorate after administration of the antitumor agent. In the present application, a skin disease or disorder associated with an antitumor agent may comprise alopecia, body odor, dermatitis bullosa, xeroderma, eczema, erythema multiforme, erythroderma, lipoatrophy, hair discolor, abnormal hair texture, hirsutism, hyperhidrosis, hyperkeratosis, hypertrichosis, hypohidrosis, lipohypertrophy, nail changes, nail discolor, nail loss, nail ridge, cutaneous pain, hand-foot syndrome, photosensitivity, pruritus, purpura, acneiform rash, maculopapule, scalp pain, adermotrophia, skin hyperpigmentation, skin hypopigmentation, calluses, skin ulcer, Stevens-Johnson syndrome, subcutaneous emphysema, capillary telangiectasia, toxic epidermal necrolysis, rash and/or urticaria. The definitions and grades of these skin diseases or disorders may refer to any version of NCI-CTCAE.

In the present application, the term “cutaneous or subcutaneous tissue adverse event” generally refers to a harmful and undesirable response, effect, action, consequence, result or influence associated with cutaneous or subcutaneous tissues or developed on cutaneous or subcutaneous tissues caused by a certain medicament or other medical treatments such as anti-cancer medicament or surgery.

In the present application, the term “associated with an antitumor agent” generally refers to that when the subject is not administered with the antitumor agent, there are not any symptoms of side effects resulting from the administration of the antitumor agent and/or no symptoms of side effects resulting from the administration of the antitumor agent can be observed, detected or diagnosed. However, when the subject is administered with the antitumor agent, at the same time as the administration or after a period (e.g., over 24 hrs, over 1 week or longer), symptoms of side effects resulting from the administration of the antitumor agent (e.g., skin diseases or disorders associated with administration of the antitumor agent) occur and/or can be observed, detected or diagnosed. In the present application, a skin disease or disorder associated with an antitumor agent may be a skin disease or disorder associated with the administration of the antitumor agent. “A skin disease or disorder associated with the administration of the antitumor agent” generally refers to a skin disease or disorder having a certain correlation with the administration of the antitumor agent to the subject.

In the present application, the term “NCI-CTCAE” generally refers to the standardized definition of adverse events as issued by National Cancer Institute (NCI)-Common Terminology Criteria for Adverse Events (CTCAE), which is used to describe the severity of organ toxicity in patients treated for cancers. As the progress of scientific evidence, such criteria may be updated constantly. In the present application, “NCI-CTCAE” may include any version of “NCI-CTCAE”.

In the present application, the term “cancer” generally refers to any medical condition, which is mediated by the growth, proliferation, or metastasis of tumors or malignant cells, and causes solid tumors or non-solid tumors. The cancers as described in the present application may comprise, but are not limited to, epithelial malignant tumor (epithelium-derived cancer), lung cancer (e.g., non-small-cell lung cancer), breast cancer, skin cancer, bladder cancer, colon cancer, intestinal cancer, prostate cancer, pancreas cancer, uterus cancer, cervical cancer, ovarian cancer, esophageal cancer, head and neck cancer, stomach cancer and laryngeal cancer.

In the present application, the term “does not substantially affect” generally mean that as compared to the therapeutic effect of using the antitumor agent alone, the use of the combination of the medicament of the present application and the antitumor agent has a comparable therapeutic effect, or does not produce a significant disadvantage, or the use of the combination of the medicament of the present application and the antitumor agent may produce a better effect (e.g., when the medicament of the present application can enhance the antitumor effect of the antitumor agent). For example, for any subject, as compared to the therapeutic effect of using the antitumor agent alone, the combination of the medicament and the antitumor agent causes the same degree of tumor volume reduction, or the degree of reduction is not less than about 5%, not less than about 4%, not less than about 3%, not less than about 2%, not less than about 1%, not less than about 0.5%, not less than about 0.1%, not less than about 0.01%, not less than about 0.001% or less, or the combination of the medicament and the antitumor agent causes tumor volume reduction at a degree of greater than about 0.001%, greater than about 0.01%, greater than about 0.1%, greater than about 0.5%, greater than about 1%, greater than about 2%, greater than about 3%, greater than about 4%, greater than about 5% or more.

### DETAILED DESCRIPTION OF THE INVENTION

Compound as shown in Formula I

In one aspect, the present application provides a use of a compound as shown in Formula I or a pharmaceutically acceptable salt, prodrug, isotopic variant, or solvate thereof in preparing a medicament, the medicament is used for preventing, relieving and/or treating a skin disease or disorder associated with an antitumor agent in a subject:

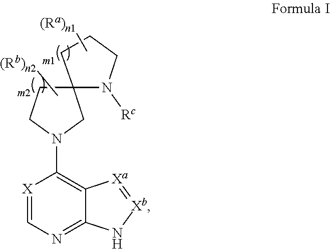

wherein,
R^{a} may be the same or different, and each may be (1) C₁₋₆ alkyl, or (2) halogen atom;
n1 may be an integer selected from 0 to 4;
R^{b} may be the same or different, and each may be (1) C₁₋₆ alkyl, or (2) halogen atom;
n2 may be an integer selected from 0 to 4;
m1 may be an integer selected from 0 to 3;
m2 may be an integer selected from 1 to 4;
X^{a}═X^{b} may be (1) CH═CH, (2) N═CH, or (3) CH═N;
X may be (1) nitrogen atom, or (2) C—R^{d}, wherein R^{d} may be hydrogen atom or halogen atom;
R^{c} may be a group selected from the following (1) to (6): (1) hydrogen atom, (2) C₁₋₆ alkyl optionally substituted by the same or different 1 to 5 substituents selected from the following Group A, (3) —C(═O)—R^{c1}, (4) —C(═O)—O—R^{c2}, (5) —C(═O)—NR^{c3}R^{c4}, wherein R^{c1}, R^{c2}, R^{c3} and R^{c4} are the same or different and each is: hydrogen atom, or (ii) C₁₋₆ alkyl optionally substituted by the same or different 1 to 5 substituents selected from the following Group A, or (6) a group with a structure of

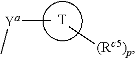

wherein,
Y^{a} may be a group selected from the following (i) to (iii): (i) C₁₋₆ alkylene, (ii) —C(═O)—, or (iii) —C(═O)—O—,
Ring T may be (i) C₆₋₁₀ aryl, (ii) C₃₋₁₀ cycloalkyl, or (iii) saturated monoheterocyclyl containing 1 to 4 heteroatoms selected from nitrogen atom, oxygen atom or sulfur atom as well as carbon atoms, and the number of the constituent ring atoms may be 3 to 7,
R^{c5} may be the same or different and each is: (i) cyanogroup, or (ii) nitro, p is an integer selected from 0 to 4;
Group A may be the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

In the present application, the term “a compound as shown in Formula I” may be known as “a compound of Formula I”, “Formula I”. Such a term is also defined as comprising all the forms of the compound of Formula I, including hydrate, solvate, isomer, crystalline and non crystalline form, isomorphism, polymorphism, and metabolite. For example, the compound of Formula I or a pharmaceutically acceptable salt thereof may be present in an unsolvated or solvated form. When the binding force of solvent or water is relatively strong, the coordination compound has a definite stoichiometry that is not affected by humidity. However, if the binding force of solvent or water is relatively weak, such as in a channel solvate and hygroscopic compound, the content of water/solvent will depend on the humidity and drying conditions, in which case non-stoichiometry is the norm.

The “compound of Formula I” may comprise chiral carbon atom(s). In the present application, the carbon-carbon bonds in the compound of Formula I may be represented by solid lines, solid wedges, or dot wedges. Depicting the bond to a chiral carbon atom with solid lines means that all the potential stereoisomers (e.g., specific enantiomers, racemates, etc.) on the carbon atom are comprised. The compound of the present application may comprise more than one chiral carbon atoms. In these compounds, depicting the bond to a chiral carbon atom with solid lines is intended to indicate that all the potential stereoisomers should be encompassed. For example, unless otherwise indicated, the compound of Formula I may present as enantiomers and diastereomers, or racemates and mixtures. Depicting the bond to one or more chiral carbon atoms in the compound of Formula I with solid lines and depicting the bond to another chiral carbon atom in the same compound with solid or dot wedges indicate the presence of a mixture of diastereomers.

The compound of the present application may be present as inclusion compounds or other coordination compounds. The present invention encompasses complexes, e.g., inclusion compounds, drug-host inclusion complex, wherein in contrast to the aforesaid solvates, drug and host are present in stoichiometric or non-stoichiometric amount. The present invention further comprises a coordination compound of Formula I comprising two or more organic and/or inorganic components that may be stoichiometric or non-stoichiometric. The resultant complex may be ionized, partially ionized, or unionized.

The stereoisomer of Formula I comprises cis- and trans-isomers, optical isomers, e.g., R and S enantiomers, diastereomers, geometrical isomers, rotamers, conformational isomers and tautomers, the compound of Formula I, including the compounds exhibiting one or more of the aforesaid types of isomerisms, and mixtures thereof (e.g., racemate and diastereomer pair). The stereoisomer further comprises acid or base addition salts in which the counter ion has optical activity, e.g., D-lactate or L-lysine, or racemates, e.g., DL-tartrate or DL-arginine.

When any racemate crystallizes, there may be two different types of crystals. The first type is the racemic compounds (true racemates) as described above, wherein a homogeneous form of crystal is produced and comprises two enantiomers in equimolar amounts. The second type is a racemic mixture or agglomerate, wherein two forms of crystals are produced in equimolar amounts, each comprising a single enantiomer.

The compound of Formula I can exhibit tautomerism and structural isomerism. For example, the compound of Formula I may be present in several tautomeric forms, comprising enol and imine forms, and ketone and enamine forms; as well as geometric isomers and their mixtures. All these tautomeric forms are encompassed within the scope of the compound of Formula I. Tautomers are present in solution as a mixture of tautomers. In the solid form, one tautomer generally dominates. Even if one tautomer may be described, the present invention also comprises all the tautomers of the compound of Formula I.

The present invention further comprises isotope-labeled compounds that are the same as that of Formula I except that one or more atoms thereof are replaced with atoms having atomic mass or mass number different from those found in nature. Isotopes which may be incorporated into the compound of Formula I comprise isotopes of hydrogen, carbon, nitrogen, oxygen, phosphorus, fluorine and chlorine, e.g., but are not limited to: ²H, ³H, ¹³C, ¹⁴C, ¹⁵N, ¹⁸O, ¹⁷O, ³¹P, ³²P, ³⁵S, ¹⁸F, and ³⁶Cl. Some isotope-labeled compounds of Formula I, e.g., into which radioisotopes (e.g., ³H and ¹⁴C) are incorporated, may be used for measuring the distribution of drugs and/or substrate tissues due to their easy preparation and detectability. Heavier isotopes, such as, ²H, can provide certain therapeutic advantages due to its greater metabolic stability, e, g., longer half-life in the body or lower dose requirements. The isotope-labeled compound of Formula I generally may be prepared with isotope-labeled reagents instead of non-isotope-labeled reagents.

The compound of the present application may be used as salts derived from inorganic or organic acids. Some compounds have advantages, such as, enhanced drug stability at different temperatures and humidities, or desired solubilities in water/oil due to the physical properties of one or more salts. In some cases, the salts of the compound can also be adjuvants for use in the separation, purification, and/or resolution of the compound.

For example, X^{a} may be carbon atom and X^{b} may be carbon atom. In some embodiments, X^{a} may be nitrogen atom and X^{b} may be carbon atom. For example, X^{a} may be carbon atom and X^{b} may be nitrogen atom,

For example, in Formula I: n1 may be an integer selected from 0 to 2; n2 may be an integer selected from 0 to 2; m1 may be an integer selected from 0 to 3; m2 may be an integer selected from 1 to 3; X may be (1) nitrogen atom, or (2) C—R^{d}, wherein R^{d} may be halogen atom; R^{c} may be a group selected from the following (1) to (6): (1) hydrogen atom, (2) CV-6 alkyl substituted by one substituent selected from the following Group A. (3) —C(═O)—R^{c1}, (4) —C(═O)—O—R^{c2}, (5) —C(═O)NR^{c3}R^{c4}, wherein R_{c1} may be C₁₋₆ alkyl optionally substituted by one substituent selected from the following Group A, R^{c2} may be C₁₋₆ alkyl, R^{c3} is C₁₋₆ alkyl optionally substituted by one substituent selected from the following Group A R^{c4} may be (i) hydrogen atom, or (ii) C₁₋₆ alkyl, or (6) a group with a structure of

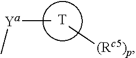

wherein, Y^{a}, may be a group selected rom the following (i) to (iii): (i) C₁₋₆ alkylene, (ii) —C(═O), or (iii) —C(═O)—O—; Ring T is (i) phenyl (ii) C₃₋₆ cycloalkyl, or (iii) pyrrolidinyl; R^{c5} may be (i) cyanogroup, or (ii) nitro, p may be an integer selected from 0 or 1; Group A may be the group consisting of (a) hydroxyl (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

For example, m1 may be an integer selected from 0 or 1, m2 is an integer selected from 1 or 2.

For example, m1 may be 1, m2 may be 2, and the compound may be a compound as shown in Formula II:

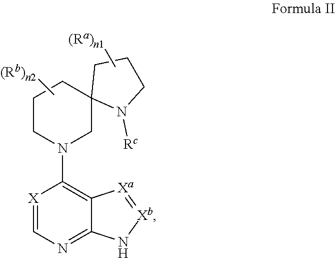

wherein, R^{a}, R^{b}, R^{c}, X, X^{a}, X^{b}, n1 and n2 may, be defined as above.

For example, m1 may be 0, m2 may be 2, and the compound may be a compound of Formula III:

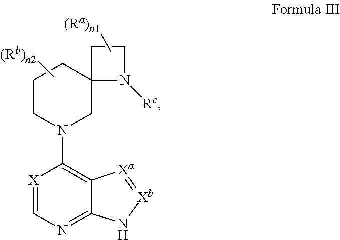

wherein Rₐ, R^{b}, R^{c}, X, X^{b}, n1 and n2 may be defined as above.

For example, m1 may be 0, m2 may be 1, and the compound may be a compound of Formula IV:

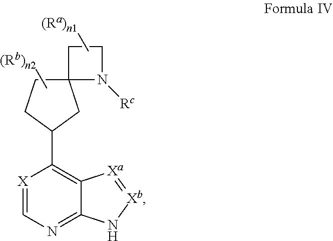

wherein R^{a} may be the same or different, and each may be (1) C₁₋₆ alkyl, or (2) halogen atom;
n1 may be an integer selected from 0 to 4,
R^{b} may be the same or different, and each may be (1) C₁₋₆ alkyl, or (2) halogen atom;
n2 may be an integer selected from 0 to 4;
X^{a}═X^{b} may be (1) CH═CH, (2) N═CH, or (3) CH═N;
X may be (1) nitrogen atom, or (2) C—R^{d}, wherein R_{d} may be hydrogen atom or halogen atom;
R^{c} may be a group selected from the following (1) to (6): (1) hydrogen atom, (2) C₁₋₆ alkyl optionally substituted by the same or different 1 to 5 substituents selected from the following Group A, (3) —C(═O)—R^{c1}, (4) —C(═O)—O—R^{c2}, (5) —C(═O)—NR^{c3}R^{c4}, wherein R^{c1}, R^{c2}, R^{c3} and R^{c4} may be the same or different, and each may be hydrogen atom, or (ii) C₁₋₆ alkyl optionally substituted by the same or different 1 to 5 substituents selected from the following Group A, or (6) a group with a structure of

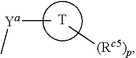

wherein,
Y^{a} may be a group selected from the following (i) to (iii): (i) C₁₋₆ alkylene, (ii) —C(═O)—, or (iii) —C(═O)—O—,
Ring T may be (i) C₆₋₁₀ aryl, (ii) C₃₋₁₀ cycloalkyl, or (iii) saturated monoheterocyclyl containing 1 to 4 heteroatoms selected from nitrogen atom, oxygen atom or sulfur atom as well as carbon atoms, and the number of the constituent ring atoms may be 3 to 7.

R^{c5} may be the same or different and each: (i) cyanogroup, or (ii) nitro, p may be an integer selected from 0 to 4;
Group A may be the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

In the present application, X^{a} may be carbon atom and X^{b} may be carbon atom. In the present application, X^{a} may be nitrogen atom and X^{b} may be carbon atom. In the present application, X^{a} may be carbon atom and X^{b} may be nitrogen atom.

In the present application, in Formula I: n1 may be an integer selected from 0 to 2; n2 may be an integer selected from 0 to 2; m1 may be an integer selected from 0) to 3; m2 may be an integer selected from 1 to 3; X may be (I) nitrogen atom, or (2) C—R^{d}, wherein R^{d} may be halogen atom; R^{c} may be a group selected from the following (1) to (6): (1) hydrogen atom, (2) C₁₋₆ alkyl substituted by one substituent selected from the following Group A, (3) —C(═O)—R^{c1}, (4) —C(═O)—O—R^{c2}, (5) —C(═O)—NR^{c3}R^{c4}, wherein R^{c1} may be C₁₋₆ alkyl optionally substituted by one substituent selected from the following Group A, R^{c2} may be C₁₋₆ alkyl, R^{c3} may be C₁₋₆ alkyl optionally substituted by one substituent selected from the following Group A, R^{c4} may be (i) hydrogen atom, or (ii) C₁₋₆ alkyl, or (6) a group with a structure of

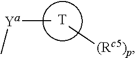

wherein, Y^{a} may be a group selected from the following (i) to (iii): (i) C₁₋₆ alkylene, (ii) —C(═O), or (iii) —C(═O)—O—; Ring T is (i) phenyl, (ii) C₃₋₆ cycloalkyl, or (iii) pyrrolidinyl; R^{c5} is (i) cyanogroup, or (ii) nitro, p is an integer selected from 0 or 1; Group A may be the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

In the present application, the R^{a} may be methyl or fluorine atom.

In the present application, n1 may be an integer of 0, 1 or 2.

In the present application, R^{b} may comprise methyl or fluorine atom.

In the present application, n2 may be an integer of 0, 1 or 2.

In the present application, m1 may be an integer selected from 0 to 3.

In the present application, m2 may be an integer of 1, 2 or 3.

In the present application, m1 may be 0 and m2 may be 1, m1 may be 0 and m2 may be 2, m1 may be 0 and m2 may be 3, m1 may be 1 and m2 may be 1, m1 may be 1 and m2 may be 2, m1 may be 2 and m2 may be 1, m1 may be 2 and m2 may be 2, or, m1 may be 3 and m2 may be 2.

In the present application, R^{a} and R^{b} may be substituted on carbon atoms other than spiro carbon of each spiro ring constituting the Formula I, and the carbon atoms not substituted by R^{a} or R^{b} are saturated with hydrogen atoms. For the case that n1 is greater than 2, R^{a} may be the same or different, and may be substituted at the same or different positions respectively. Further, for the case that n2 is greater than 2, R^{b} may be the same or different, and may be substituted at the same or different positions respectively.

In the present application, the m1 and m2 may be selected from the following group: (1) m1 is 0, m2 is 3, (2) m1 is 2, m2 is 1, (3) m1 is 2, m2 is 2, and (4) m1 is 3, m2 is 2.

In the present application, X^{a}═X^{b} is CH═CH, X is nitrogen atom. In the present application, X^{a} is carbon atom, X^{b} is carbon atom, X is nitrogen atom.

In the present application, n1 may be 0, and n2 may be 0. In the present application, n1 may be 1, and n2 may be 0. In the present application, n1 may be 0, and n2 may be 1. In the present application, n1 may be 2, and n2 may be 0. In the present application, n1 may be 0, and n2 may be 2.

In the present application, R^{a} may be methyl or fluorine atom.

In the present application, R^{c} may be —C(═O)—R^{c1}. In the present application, R^{c} may be C₁₋₆ alkyl substituted by one hydroxyl or cyanogroup. In the present application, R^{c} may be —C(═O)—R^{c3}R^{c4}.

In the present application, R^{c3} may be C₁₋₆ alkyl substituted by one cyanogroup, R^{c4} is hydrogen.

In the present application, the compound as shown in Formula I may be selected from the following group:

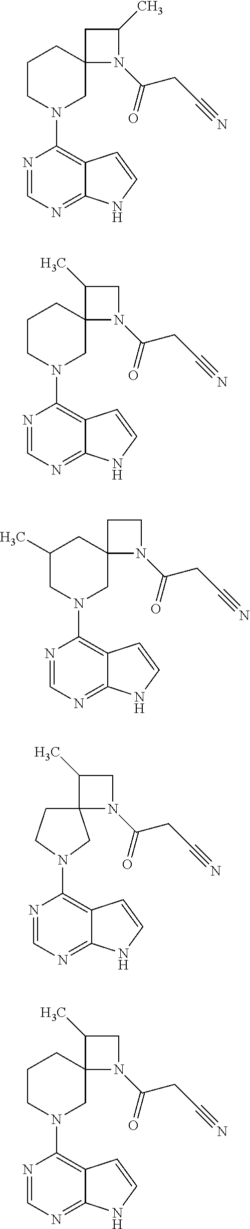 [SMILES: CC1CC2(CCCN(c3ncnc4[nH]ccc34)C2)N1C(=O)CC#N ; CC1CN(C(=O)CC#N)C12CCCN(c1ncnc3[nH]ccc13)C2 ; CC1CN(c2ncnc3[nH]ccc23)CC2(CCN2C(=O)CC#N)C1 ; CC1CN(C(=O)CC#N)C12CCN(c1ncnc3[nH]ccc13)C2 ; CC1CN(C(=O)CC#N)C12CCCN(c1ncnc3[nH]ccc13)C2]

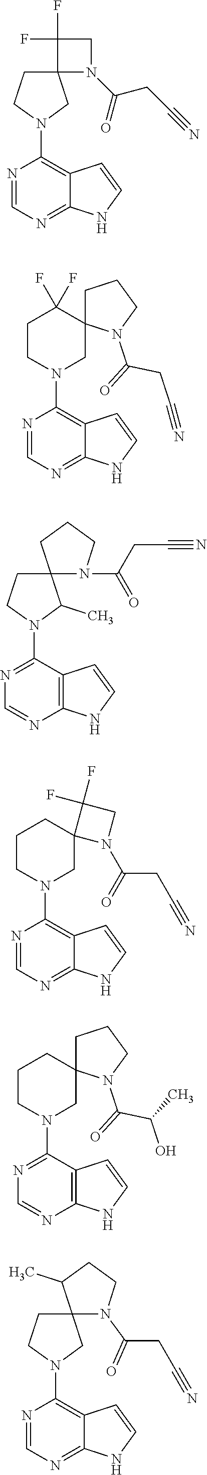 [SMILES: N#CCC(=O)N1CC(F)(F)C12CCN(c1ncnc3[nH]ccc13)C2 ; N#CCC(=O)N1CCCC12CN(c1ncnc3[nH]ccc13)CCC2(F)F ; CC1N(c2ncnc3[nH]ccc23)CCC12CCCN2C(=O)CC#N ; N#CCC(=O)N1CC(F)(F)C12CCCN(c1ncnc3[nH]ccc13)C2 ; C[C@H](O)C(=O)N1CCCC12CCCN(c1ncnc3[nH]ccc13)C2 ; CC1CCN(C(=O)CC#N)C12CCN(c1ncnc3[nH]ccc13)C2]

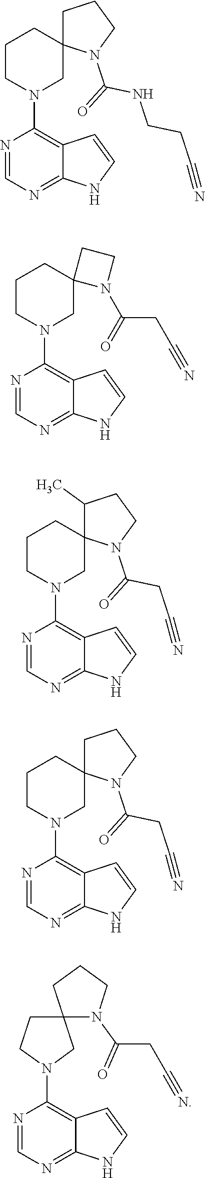 [SMILES: N#CCCNC(=O)N1CCCC12CCCN(c1ncnc3[nH]ccc13)C2 ; N#CCC(=O)N1CCC12CCCN(c1ncnc3[nH]ccc13)C2 ; CC1CCN(C(=O)CC#N)C12CCCN(c1ncnc3[nH]ccc13)C2 ; N#CCC(=O)N1CCCC12CCCN(c1ncnc3[nH]ccc13)C2 ; N#CCC(=O)N1CCCC12CCN(c1ncnc3[nH]ccc13)C2]

In the present application, the compound as show in in Formula I may be selected from the following group:

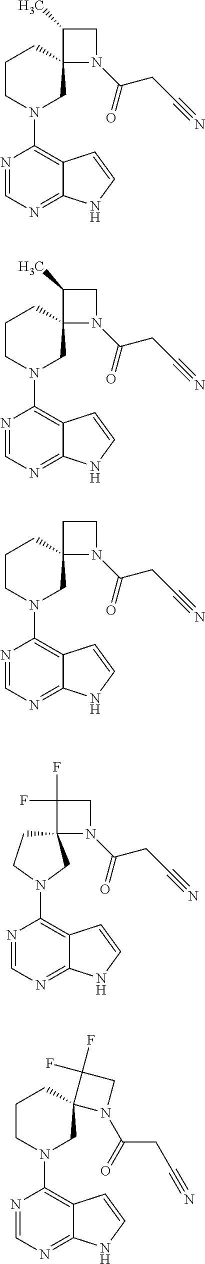 [SMILES: C[C@@H]1CN(C(=O)CC#N)[C@]12CCCN(c1ncnc3[nH]ccc13)C2 ; C[C@H]1CN(C(=O)CC#N)[C@]12CCCN(c1ncnc3[nH]ccc13)C2 ; N#CCC(=O)N1CC[C@@]12CCCN(c1ncnc3[nH]ccc13)C2 ; N#CCC(=O)N1CC(F)(F)[C@@]12CCN(c1ncnc3[nH]ccc13)C2 ; N#CCC(=O)N1CC(F)(F)[C@@]12CCCN(c1ncnc3[nH]ccc13)C2]

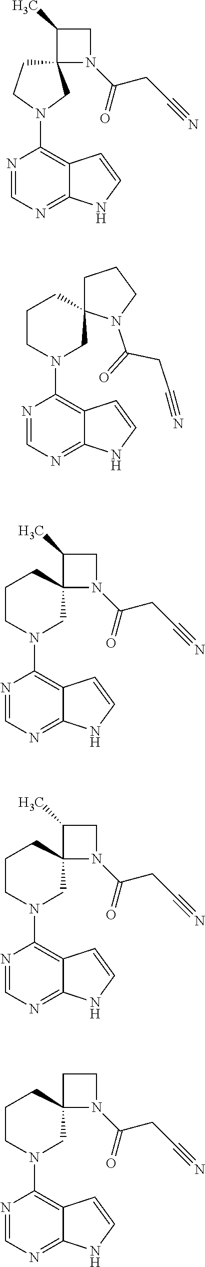 [SMILES: C[C@H]1CN(C(=O)CC#N)[C@]12CCN(c1ncnc3[nH]ccc13)C2 ; N#CCC(=O)N1CCC[C@@]12CCCN(c1ncnc3[nH]ccc13)C2 ; C[C@H]1CN(C(=O)CC#N)[C@@]12CCCN(c1ncnc3[nH]ccc13)C2 ; C[C@@H]1CN(C(=O)CC#N)[C@@]12CCCN(c1ncnc3[nH]ccc13)C2 ; N#CCC(=O)N1CC[C@]12CCCN(c1ncnc3[nH]ccc13)C2]

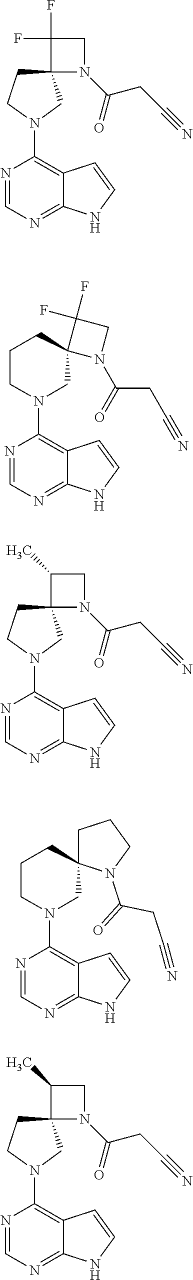 [SMILES: N#CCC(=O)N1CC(F)(F)[C@]12CCN(c1ncnc3[nH]ccc13)C2 ; N#CCC(=O)N1CC(F)(F)[C@]12CCCN(c1ncnc3[nH]ccc13)C2 ; C[C@@H]1CN(C(=O)CC#N)[C@@]12CCN(c1ncnc3[nH]ccc13)C2 ; N#CCC(=O)N1CCC[C@]12CCCN(c1ncnc3[nH]ccc13)C2 ; C[C@H]1CN(C(=O)CC#N)[C@@]12CCN(c1ncnc3[nH]ccc13)C2]

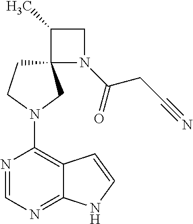 [SMILES: C[C@@H]1CN(C(=O)CC#N)[C@]12CCN(c1ncnc3[nH]ccc13)C2]

In the present application, the compound as shown in Formula I may be

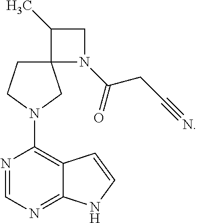 [SMILES: CC1CN(C(=O)CC#N)C12CCN(c1ncnc3[nH]ccc13)C2]

### Antitumor Agent

In the present application, the antitumor agent may comprise a small molecular compound, a small molecular conjugate, protein (e.g., antibody) and/or polynucleotide (e.g., DNA or RNA). In the present application, the antitumor agent may be a targeted therapeutic agent.

In the present application, the targeted therapeutic agent may target a tumor antigen. In some embodiments, the tumor antigen is a cell surface receptor selected from transforming growth factor receptor (TGFR), epidermal growth factor receptor (EGFR), insulin-like growth factor receptor (IGFR), fibroblast growth factor receptor (FGFR), modulin receptor, platelet derived growth factor receptor (PDGFR), vascular endothelial growth factor receptor (VEGFR) and hypoxia-inducible factor receptor (HIFR).

In the present application, the targeted therapeutic agent targets growth factor, hormone or extracellular matrix molecules. In some embodiments, the targeted therapeutic agent targets growth factor, hormone or extracellular matrix molecules selected from the following group: transforming growth factor (TGF), epidermal growth factor (EGF), insulin-like growth factor (IGF), fibroblast growth factor (FGF), modulin, platelet derived growth factor (PDGF), vascular endothelial growth factor (VEGF), hypoxia-inducible factor (HIF), c-Met, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, collagen, elastin, biglycan, decorin, lumican, versican, perlecan, C-reactive protein, ApoE and laminin.

In the present application, the targeted therapeutic agent targets a tumor antigen selected from the following group: EGFR mutant, HER2/neu, HER3, HER4, CD4, CD19, CD20, CD22, CD29b, CD30, CD33, CD37, CD38, CD52, CD70, CD79b, CD123, CD138, CD200, CD276, CXCR3, CXCR5, CCR3, CCR4, CCR9, CRTH2, PMCH, endoplasmic reticulin, CS1, CEA, mesothelin, G250, MUC1, MUC16, PSMA, ADAM17, EPCAM, EphA2, MCSP, GPA33, NAPi2b, STEAP1, CEACAM1, CEACAM5, GPNMB and TROP.

In the present application, the antitumor agent may be a tyrosine kinase inhibitor. In the present application, the antitumor agent may be a small molecular tyrosine kinase inhibitor, a protein macromolecule that binds specifically to tyrosine kinase, a RNAi that inhibits the expression of tyrosine kinase, a tyrosine kinase capture agent, a reagent that reduces the expression capacity of tyrosine kinase and/or an antisense oligonucleotide that inhibits the expression of tyrosine kinase.

In the present application, the targeted therapeutic agent may target protein and/or nucleic acid molecules within tumor cells, on tumor cell surface and/or outside tumor cells. In the present application, the targeted therapeutic agent may target one or more targets selected from the following group: EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, CD20, PD-L1 and/or BRCA1/2, or mutants thereof.

In the present application, the targeted therapeutic agent may comprise hormonotherapy, signal transduction inhibitor, gene expression modulator, cell apoptosis inducer, angiogenesis inhibitor and/or toxin delivery molecules. Hormonotherapy works by preventing the human body from producing hormones or interfering with the effects of hormones. Signal transduction inhibitor can block the activity of molecules involved in signaling. Gene expression regulator can modify the function of protein that works in the expression of gene. Apoptosis inducer can cause cancer cells to undergo a controlled cell death process that is called apoptosis. Angiogenesis inhibitor can prevent the growth of new blood vessels into tumor (tumor angiogenesis).

In the present application, the targeted therapeutic agent may target protein and/or gene selected from the following group, or mutants thereof: EGFR, MEK, ALK, BTK, PI3K, AKT, VEGFR, mTOR, HDAC, KIT, FGFR, FAK, BCRP and/or SRC, as well as a combination thereof. In the present application, the targeted therapeutic agent may be EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor and/or SRC inhibitor. In the present application, the targeted therapeutic agent may be EGFR antibody, MEK antibody, ALK antibody, BTK antibody, PI3K antibody, AKT antibody, VEGFR antibody, mTOR antibody, HDAC antibody, KIT antibody, FAK antibody, FGFR antibody, BCRP antibody and/or SRC antibody.

In the present application, the targeted therapeutic agent may be a multi-target inhibitor. In the present application, the targeted therapeutic agent may target two or more targets simultaneously. For example, the targeted therapeutic agent may target EGFR and cMET simultaneously, target ALK and EGFR simultaneously, target BCR and ABL simultaneously, target VEGFR, EGFR and ABL simultaneously, target EGFR, FGFR and VEGFR simultaneously, target VEGFR, FGFR and c-KIT simultaneously, target PI3K and BCRP simultaneously, or, target BRAF and VEGFR simultaneously. For example, the targeted therapeutic agent may be EGFR/cMET inhibitor. The EGFR/cMET inhibitor targets and inhibits EGFR and cMET simultaneously.

In the present application, the targeted therapeutic agent may be selected from the following group: afatinib, dacomitinib, osimertinib, EAI045, gefitinib, almonertinib, pyrotinib, brigatinib, neratinib, olmutinib, bosutinib, icotinib, vandetanib, lapatinib, alflutinib, BPI-7711, mobocertinib, dovitinib, zorifertinib, varlitinib, orelabrutinib, tirabrutinib, zanubrutinib, acalabrutinib, ibrutinib, dasatinib, pirtobrutinib, tolebrutinib, rilzabrutinib, fenebrutinib, evobrutinib, selumetinib, binimetinib, cobimetinib, trametinib, regorafenib, GSK-1120212, alpelisib, duvelisib, copanlisib, idelalisib, nortriptyline, inavolisib, dactolisib, apitolisib, parsaclisib, buparlisib, rigosertib, enzastaurin, paxalisib, leniolisib, ipatasertib, zotarolimus, sirolimus, everolimus, temsirolimus, sorafenib, apatinib, lenvatinib, sunitinib, cabozantinib, axitinib, nintedanib, brivanib, vatalanib, fruquintinib, dabrafenib, vemurafenib, encorafenib, pazopanib, crizotinib, panobinostat, erlotinib, rituximab, panitumumab, cetuximab, ticilimumab, erfonrilimab, BA-3071, MEDI-5752, defactinib, zalifrelimab, cadonilimab, BCD-217, ipilimumab, tremelimumab, quavonlimab, atezolizumab, durvalumab, camrelizumab, tislelizumab, sintilimab, toripalimab, pembrolizumab, nivolumab, amivantamab, MCLA-129, EMB-01, LY3164530, Roche Glycart anti-EGFR/cMet, Genentech Anti-met/EGFR, Samsung Anti-EGFR/cMet, Merck serono Anti-cMet/EGFR, GB263 and lazertinib, as well as a combination thereof.

In the present application, the antitumor agent may be an immunotherapeutic agent. In the present application, the immunotherapeutic agent can kill tumor cells by use of the immune system. In the present application, the immunotherapeutic agent may be an antibody capable of specifically recognizing and/or binding to specific molecules (e.g., target molecules) on the surface of tumor cells. The binding of a monoclonal antibody to target molecules causes immune damages to cells expressing the target molecules. In the present application, the immunotherapeutic agent may be an antibody capable of specifically recognizing and/or binding to immune cells (e.g., T cells, B cells, NK cells), which can bind to immune cells to help these cells better kill tumor cells. A monoclonal antibody delivering toxic molecules can specifically cause the death of tumor cells. Once an antibody binds to a target cell, the toxic molecules (e.g., radioactive substances or toxic chemical substances) attached to the antibody would be absorbed by the cell, and finally killing the cell. Toxins will not affect cells that lack antibody targets, i.e., most cells in the body. Antibodies, cancer vaccines and gene therapies that specifically recognize or bind to tumor antigens are sometimes considered as targeted therapies, because they interfere with the growth of specific cancer cells.

For example, the immunotherapeutic agent of the present application may be a reagent targeting the immune checkpoint. For example, the immunotherapeutic agent of the present application may be an immune checkpoint inhibitor. For example, the immunotherapeutic agent of the present application may be PD-L1 inhibitor, PD-1 inhibitor, LAG-3 inhibitor, TIM-3 inhibitor and/or CTLA-4 inhibitor.

In the present application, the immunotherapeutic agent may be an antibody, e.g., a monoclonal antibody. In the present application, the immunotherapeutic agent may be anti-PD-L1 antibody, anti-PD-1 antibody, anti-LAG-3 antibody, anti-TIM-3 antibody and/or anti-CTLA-4 antibody.

In the present application, the immunotherapeutic agent may be ipilimumab, pembrolizumab and nivolumab, as well as a combination thereof.

In the present application, the antitumor agent (e.g., the immunotherapeutic agent and/or the targeted therapeutic agent) can be administered alone.

In the present application, the antitumor agent (e.g., the immunotherapeutic agent and/or the targeted therapeutic agent) may be administered in combination with one or more other therapies. In some embodiments, the other therapies comprise surgery, radiotherapy and/or chemotherapy and other antitumor therapies.

### Skin Disease or Disorder

In the present application, the compound as shown in Formula I can prevent, relieve and/or treat a skin disease or disorder (e.g., rash) associated with an antitumor agent in a subject. In the present application, the skin disease or disorder associated with an antitumor agent may mean that the skin disease or disorder is caused by administration of the antitumor agent, which develops or deteriorates after administration of the antitumor agent. In the absence of prevention or treatment, the skin disease or disorder will occur or deteriorate about 1 hr, about 2 hrs, about 3 hrs, about 4 hrs, about 5 hrs, about 6 hrs, about 7 hrs, about 8 hrs, about 9 hrs, about 10 hrs, about 11 hrs, about 12 hrs, about 1 day, about 2 days, about 4 days, about 7 days, about 2 weeks, about 3 weeks, about 1 month, about 2 months or longer after the administration of the antitumor agent.

For example, the subject has suffered from the skin disease or disorder before the administration of the antitumor agent, and the severity grading of the skin disease or disorder increases after the administration of the antitumor agent. For example, the increase of the severity grading may mean that the severity grading of the skin disease or disorder (e.g., rash) increases from Grade 1 to Grade 2, from Grade 1 to Grade 3, from Grade 1 to Grade 4, from Grade 1 to Grade 5, from Grade 2 to Grade 3, from Grade 2 to Grade 4, from Grade 2 to Grade 5, from Grade 3 to Grade 4, from Grade 3 to Grade 5, or from Grade 4 to Grade 5, as evaluated according to CTCAE, V5.0.

For example, the subject does not suffer from the skin disease or disorder before the administration of the antitumor agent, but suffers from the skin disease or disorder after the administration of the antitumor agent. For example, as evaluated according to the criteria of CTCAE V5.0, the severity grading of the skin disease or disorder of the subject is Grade 1, Grade 2, Grade 3, Grade 4 or Grade 5.

The skin disease or disorder may comprise cutaneous or subcutaneous tissue disease, the evaluation criterion for which can refer to CTCAE V5.0.

In the present application, the skin disease or disorder may comprise alopecia, body odor, dermatitis bullosa, xeroderma, eczema, erythema multiforme, erythroderma, lipoatrophy, hair discolor, abnormal hair texture, hirsutism, hyperhidrosis, hyperkeratosis, hypertrichosis, hypohidrosis, lipohypertrophy, nail changes, nail discolor, nail loss, nail ridge, cutaneous pain, hand-foot syndrome, photosensitivity, pruritus, purpura, acneiform rash, maculopapule, scalp pain, adermotrophia, skin hyperpigmentation, skin hypopigmentation, calluses, skin ulcer, Stevens-Johnson syndrome, subcutaneous emphysema, capillary telangiectasia, toxic epidermal necrolysis, rash and/or urticaria. For example, the skin disease or disorder may be rash.

In the present application, the skin disease or disorder may comprise antitumor agent-associated alopecia, antitumor agent-associated body odor, antitumor agent-associated dermatitis bullosa, antitumor agent-associated xeroderma, antitumor agent-associated eczema, antitumor agent-associated erythema multiforme, antitumor agent-associated erythroderma, antitumor agent-associated lipoatrophy, antitumor agent-associated hair discolor, antitumor agent-associated abnormal hair texture, antitumor agent-associated hirsutism, antitumor agent-associated hyperhidrosis, antitumor agent-associated hyperkeratosis, antitumor agent-associated hypertrichosis, antitumor agent-associated hypohidrosis, antitumor agent-associated lipohypertrophy, antitumor agent-associated nail changes, antitumor agent-associated nail discolor, antitumor agent-associated nail loss, antitumor agent-associated nail ridge, antitumor agent-associated cutaneous pain, antitumor agent-associated hand-foot syndrome, antitumor agent-associated photosensitivity, antitumor agent-associated pruritus, antitumor agent-associated purpura, antitumor agent-associated acneiform rash, antitumor agent-associated maculopapule, antitumor agent-associated scalp pain, antitumor agent-associated adermotrophia, antitumor agent-associated skin hyperpigmentation, antitumor agent-associated skin hypopigmentation, antitumor agent-associated calluses, antitumor agent-associated skin ulcer, antitumor agent-associated Stevens-Johnson syndrome, antitumor agent-associated subcutaneous emphysema, antitumor agent-associated capillary telangiectasia, antitumor agent-associated toxic epidermal necrolysis, and antitumor agent-associated rash and/or urticaria. For example, the antitumor agent-associated skin disease or disorder may be antitumor agent-associated rash.

For example, the pathological manifestations of rash may comprise obvious changes in the growth and/or differentiation of skin epidermis, changes in the terminal differentiation of keratinocytes, dense orthokeratology and epidermal parakeratosis that can be observed in both affected and unaffected skin, damages in sebaceous gland and/or follicular infundibulum, with or without evidence of infection, damages in epidermal barrier, epidermal subcomeal fissure, production of cytokines, infiltration of inflammatory cells (e.g., neutrophils, lymphocytes), bacterial infection, capillary telangiectasia, pigmentation and/or dense epithelium inflammatory permeability.

For example, the clinical manifestations of rash may be erythema, xeroderma, pruritus, scaly patches, tenderness, burning sensation, cracks, pustule, follicle, ulcer, abscess, red bumps and/or purulent lesions.

For example, the occurrence site of rash may be epidermis, e.g., including seborrheic areas of skin. For example, the occurrence site of rash may comprise scalp, face, neck, chest, upper back, limbs, lower back, belly, hip, periodontal area, belly, palm, soles, nails and/or mucosa.

The severity grading of rash may be graded according to the Common Adverse Event Terminology Criteria (CTCAE) issued by U.S. National Cancer Institute, which is the standard classification and severity grading criteria for adverse events in cancer treatment clinal trials and other oncology settings (NCI-CTCAE V5.0). In some embodiments, the severity grading of epithelial diseases may be Grade 1 or above, Grade 2 or above, Grade 3 or above, Grade 4 or above, or Grade 5, as evaluated in accordance with NCI-CTCAE V5.0. The severity of skin disease or disorder may depend on the type and dosage of the antitumor agent.

The severity grading of rash may also be graded according to the American Society of Clinical Oncology (ASCO) Clinical Practice Guideline (referring to https://www.ncbi.nlm.nih.gov/pmc/articles/PMC6481621/), which classifies rash (e.g., rash caused by the immunotherapeutic agent) into 4 grades. In some embodiments, the severity of epithelial diseases may be Grade 1 or above, Grade 2 or above, Grade 3 or above, or Grade 4, as evaluated in accordance with ASCO Guideline. The severity of skin diseases may depend on the type and dosage of the antitumor agent (e.g., the immunotherapeutic agent).

In the present application, after administration of the compound as shown in Formula I of the present application, the severity of a skin disease or disorder associated with an antitumor agent in a subject can be relieved. In the present application, the relief generally means that the onset or development of the skin disease or disorder in the subject is delayed. In the present application, after administration of the compound as shown in Formula I, the symptoms of the skin disease or disorder in the subject can be alleviated. For example, as evaluated in accordance with the criterion of NCI-CTCAE, after administration of the compound as shown in Formula I, the symptom of the skin disease or disorder (e.g., rash) in the subject can be reduced from Grade 5 to Grade 4, from Grade 5 to Grade 3, from Grade 5 to Grade 2, from Grade 5 to Grade 1, from Grade 4 to Grade 3, from Grade 4 to Grade 2, from Grade 4 to Grade 1, from Grade 3 to Grade 2, from Grade 3 to Grade 1, or from Grade 2 to Grade 1. For example, in accordance with ASCO Guideline, after administration of the compound as shown in Formula I, the symptom of the skin disease (e.g., rash) in the subject can be reduced from Grade 4 to Grade 3, from Grade 4 to Grade 2, from Grade 4 to Grade 1, from Grade 3 to Grade 2, from Grade 3 to Grade 1, or from Grade 2 to Grade 1.

In the present application, after administration of the compound as shown in Formula I, the symptom of the skin disease or disorder in the subject may be eliminated. However, the case that the skin disease or disorder may recur or worsen again after discontinuation of the compound as shown in Formula I is not excluded.

In the present application, the skin disease or disorder may include cutaneous or subcutaneous tissue diseases associated with single administration of an antitumor agent.

In the present application, the skin disease or disorder may include cutaneous or subcutaneous tissue diseases associated with combined administration of two or more antitumor agents.

In the present application, the skin disease or disorder may include skin diseases and subcutaneous tissue diseases associated with administration of an antitumor agent in combination with one or more other therapies. In some embodiments, the other therapies comprise surgery, radiotherapy and/or chemotherapy, and other antitumor therapies.

### Therapeutic Method and Composition

The term “prevention” as used herein generally refers to the prevention of onset, recurrence, or spread of diseases or one or more symptoms thereof. In the context of the present application, the “prevention” may be interchangeably used with the “preventive treatment”. In some embodiments, the “prevention” generally refers to the treatment of providing a patient suffering from the diseases or disorders as described in the present application with the medicament in accordance with the present application with or without administration of other medicaments as described in the present application prior to the onset of any symptoms. In some embodiments, the patients with a family history of a particular disease may be deemed as candidates of the preventive regimen. In some embodiments, the patients having a history of symptom recurrence are also potential subjects of prevention.

The term “treatment” as used herein generally refers to eliminate or ameliorate the disease, or one or more symptoms associated with the disease. In some embodiments, the treatment generally refers to eliminate or ameliorate a disease by administering one or more therapeutic agents to the patient with such a disease. In some embodiments, the “treatment” may be administering a medicament in the presence or absence of other therapeutic agent(s) after the onset of the symptoms of a particular disease.

The term “subject” as used herein generally refers to a human or non-human animal (including mammal) in need of diagnosing, prognosing, improving, preventing and/or treating diseases, especially those in need of treatment, amelioration or prevention by using the compound as shown in Formula I. In some embodiments, the subject may comprise a cancer patient. For example, the cancer patient has been, is being, and/or will be administered with the antitumor agent.

In some embodiments, the subject may be a human or non-human mammal. The non-human mammal may include any mammalian species other than human, e.g., livestocks (e.g., cow, pig, sheep, chick, rabbit or horse), or rodents (e.g., rat and mouse), or primates (e.g., gorilla and monkey), or domestic animals (e.g., dog and cat). The “subject” may be male or female, and may also be at different ages.

The term “effective amount” as used herein generally refers to an amount of medicament capable of ameliorating or eliminating diseases or symptoms of the subject, or prophylactically inhibiting or preventing the onset of diseases or symptoms. The effective amount may be an amount of medicament capable of ameliorating one or more diseases or symptoms to a certain extent in the subject; an amount of medicament capable of partially or completely restoring one or more physiological or biochemical parameters associated with the causes of diseases or symptoms to normal; and/or an amount of medicament capable of reducing the possibility of the occurrence of diseases or symptoms.

In the present application, the administration site of the compound as shown in Formula I may not be the occurrence site of cancer or potential metastatic site of cancer. For example, the administration site may not be the primary site of cancer. As another example, the administration site may not be a metastatic site of cancer. For example, the metastatic site may comprise sites of cancer metastasis occurrence resulting from lymphatic metastasis, vascular metastasis, and/or implantative metastasis. In some embodiments, the metastatic site may comprise bone, brain, liver, stomach, and/or lung. As another example, the administration site may not be a recurrence site of cancer.

The compound as shown in Formula I of the present application may be administered in a manner well known in the art, e.g., by injection (e.g., subcutaneous, intraperitoneal, intraarticular, intraarterial, intrathecal, intrasternal, intrathecal, intralesional, intracranial, intramuscular, intracutaneous and intravenous injection or infusion) or non-injection (e.g., oral, nasal, sublingual, vaginal, rectal, or topical administration). The compound as shown in Formula I of the present application may be administered in a form of a pharmaceutical composition or a kit. In some embodiments, the compound as shown in Formula I of the present application may be administered in the same administration route as the antitumor agent or administered in a different route. In some embodiments, the medicament or the compound as shown in Formula I is prepared for dermal local administration. For example, in the present application, the medicament or the compound as shown in Formula I may be prepared as cream, lotion, gel, ointment, oleamen, spray, liposome preparation, liniment and/or aerosol. For example, in the present application, the transdermal dosage forms prepared from the medicament or the compound as shown in Formula I may be transdermal preparations in solution (cream, gel, ointment, paste, etc.), and may also be transdermal preparations in suspension (cream, gel, ointment, paste, etc.).

In the present application, the medicament and/or the compound as shown in Formula I may be prepared for oral administration. For example, the compound as shown in Formula I may be administered transdermally and/or locally administration, the antitumor agent may be administered orally or by injection.

In some embodiments, the compound as shown in Formula I of the present application may be administered together with the antitumor agent. In some embodiments, the compound as shown in Formula I may be administered before, simultaneously with, or after the administration of the antitumor agent to the subject. In some embodiments, the compound as shown in Formula I may be separately administered from the antitumor agent as a part of a multi-dose regimen. In some embodiments, the compound as shown in Formula I may be simultaneously administered with the antitumor agent. In the embodiments of simultaneous administration, these compounds as shown in Formula I may be a part of a single dosage form, which is mixed with the currently disclosed antitumor agent to form a single composition. In other embodiments, these compounds as shown in Formula I may be approximately simultaneously administered with the antitumor agent as a separate dose.

In the embodiments in which the compound as shown in Formula I and the antitumor agent are administered at intervals, the compound as shown in Formula I may be administered before or after the administration of the antitumor agent at intervals. The time intervals may be 1 min, 2 min, 5 min, 10 min, 20 min, 30 min, 45 min, 1 hr, 2 hrs, 3 hrs, 4 hrs, 5 hrs, 6 hrs, 12 hrs, 18 hrs, 1 day, 2 days, 3 days, 1 week, 2 weeks, 3 weeks, 1 month, 2 months, 3 months or longer.

In some embodiments, the compound as shown in Formula I or a pharmaceutically acceptable salt, prodrug, isotopic variant, or solvate thereof may be administered as a medicament or a part of a pharmaceutical combination. In some embodiments, the medicament may comprise the compound as shown in Formula I or a pharmaceutically acceptable salt, prodrug, isotopic variant, or solvate thereof and one or more pharmaceutically acceptable carriers.

In some embodiments, the pharmaceutical composition or the kit may comprise 1) the antitumor agent; and 2) the compound as shown in Formula I or a pharmaceutically acceptable salt, or a solvate thereof. In some embodiments, the antitumor agent and the compound as shown in Formula I or a pharmaceutically acceptable salt, prodrug, isotopic variant, or solvate thereof may not be mixed with each other. For example, the antitumor agent and the compound as shown in Formula I or a pharmaceutically acceptable salt, prodrug, isotopic variant, or solvate thereof may be each independently present in a separate container. For example, the antitumor agent may be packaged in one reagent bottle, and the compound as shown in Formula I or a pharmaceutically acceptable salt, or solvate thereof may be packaged in another reagent bottle.

In the pharmaceutical combination or kit of the present application, the compound as shown in Formula I or a pharmaceutically acceptable salt, prodrug, isotopic variant, or solvate thereof as described in 2) can prevent or treat the disease or disorder caused by the antitumor agent as described in 1). In the pharmaceutical combination or kit of the present application, the compound as shown in Formula I or a pharmaceutically acceptable salt, prodrug, isotopic variant, or solvate thereof as described in 2) does not substantially affect the therapeutic effect of the antitumor agent as described in 1).

In the present application, the administration frequency of the compound as shown in Formula I or a pharmaceutically acceptable salt, prodrug, isotopic variant, or solvate thereof may be the same as or different that of the antitumor agent.

In the present application, the concentration of the compound as shown in Formula I may be about 0.0001% (w/w) to about 50% (w/w), for example, may be about 0.001% (w/w) to about 40% (w/w), about 0.001% (w/w) to about 30% (w/w), about 0.001% (w/w) to about 20% (w/w), about 0.001% (w/w) to about 10% (w/w), about 0.01% (w/w) to about 9% (w/w), about 0.01% (w/w) to about 8% (w/w), about 0.01% (w/w) to about 7% (w/w), about 0.01% (w/w) to about 6% (w/w), about 0.01% (w/w) to about 5% (w/w), about 0.01% (w/w) to about 4% (w/w), about 0.01% (w/w) to about 3% (w/w), about 0.01% (w/w) to about 2% (w/w), about 0.01% (w/w) to about 1% (w/w), about 0.01% (w/w) to about 0.5% (w/w), about 0.01% (w/w) to about 0.1% (w/w) or about 0.01% (w/w) to about 0.05% (w/w). As another example, the concentration of the compound as shown in Formula I in the present application may be about 0.01% (w/w) to about 0.05% (w/w), about 0.01% (w/w) to about 1% (w/w), about 0.01% (w/w) to about 2% (w/w), about 0.01% (w/w) to about 5% (w/w), about 0.05% (w/w) to about 1% (w/w), about 1% (w/w) to about 2% (w/w), or about 2% (w/w) to about 5% (w/w), or about 0.2% (w/w) to about 5% (w/w).

In the present application, the medicament may further include one or more pharmaceutically acceptable carriers. The pharmaceutically acceptable carriers may include but not limited to, e.g., pharmaceutically acceptable liquid, gel or solid carriers, aqueous medium, non-aqueous medium, antimicrobial substances, isotonic substances, buffer solution, antioxidants, anaesthetics, suspending agents/dispersing agents, chelating agents, emulsifiers, diluents, adjuvants, accessories, non-toxic auxiliary substances, fillers, binders, disintegrants, buffer solution, preservatives, lubricants, flavoring agents, thickening agents, colorants, emulsifiers, other components well known in the art or various combinations of the above.

In another aspect, the present application provides a method of preventing, relieving and/or treating a skin disease or disorder associated with an antitumor agent, comprising administering to a subject in need thereof the compound as shown in Formula I of the present application or a pharmaceutically acceptable salt, prodrug, isotopic variant, or solvate thereof.

In another aspect, the present application provides a method of preventing, relieving and/or treating a skin disease or disorder associated with an antitumor agent, comprising administering

to a subject in need thereof the compound

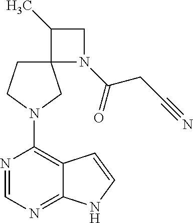 [SMILES: CC1CN(C(=O)CC#N)C12CCN(c1ncnc3[nH]ccc13)C2]

or a pharmaceutically acceptable salt, prodrug, isotopic variant, or solvate thereof. For example, the skin disease or disorder may be rash.

In another aspect, the present application provides the compound as shown in Formula I of the present application or a pharmaceutically acceptable salt, prodrug, isotopic variant, or solvate thereof, which is used for preventing, relieving and/or treating a skin disease or disorder associated with an antitumor agent.

In another aspect, the present application provides a compound

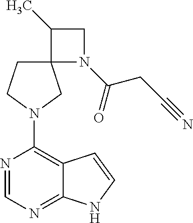 [SMILES: CC1CN(C(=O)CC#N)C12CCN(c1ncnc3[nH]ccc13)C2]

or a pharmaceutically acceptable salt, prodrug, isotopic variant, or solvate thereof which is used for preventing, relieving and/or treating a skin disease or disorder associated with an antitumor agent. For example, the skin disease or disorder may be rash.

In another aspect, the present application provides the compound as shown in Formula I or a pharmaceutically acceptable salt, prodrug, isotopic variant, or solvate thereof, which is used for preventing, relieving and/or treating a skin disease or disorder associated with an antitumor agent in a subject. For example, the skin disease or disorder may be rash.

In another aspect, the present application provides a use of

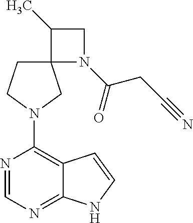 [SMILES: CC1CN(C(=O)CC#N)C12CCN(c1ncnc3[nH]ccc13)C2]

or a pharmaceutically acceptable salt, prodrug, isotopic variant, or solvate thereof in preparing a medicament, the medicament is used for preventing, relieving and/or treating a skin disease or disorder associated with an antitumor agent in a subject. For example, the skin disease or disorder may be rash.

In another aspect, the present application provides a pharmaceutical combination, comprising the compound as shown in Formula I of the present application or a pharmaceutically acceptable salt, prodrug, isotopic variant, or solvate thereof and an antitumor agent.

In another aspect, the present application provides a pharmaceutical composition, comprising the compound

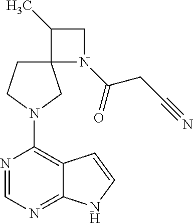 [SMILES: CC1CN(C(=O)CC#N)C12CCN(c1ncnc3[nH]ccc13)C2]

or a pharmaceutically acceptable salt, prodrug, isotopic variant, or solvate thereof and an antitumor agent. For example, the skin disease or disorder may be rash.

In another aspect, the present application provides a kit, comprising the compound as shown in Formula I of the present application or a pharmaceutically acceptable salt, prodrug, isotopic variant or solvate thereof and an antitumor agent.

In another aspect, the present application provides a kit, comprising the compound

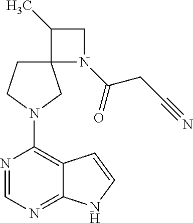 [SMILES: CC1CN(C(=O)CC#N)C12CCN(c1ncnc3[nH]ccc13)C2]

or a pharmaceutically acceptable salt, prodrug, isotopic variant, or solvate thereof and an antitumor agent. For example, the skin disease or disorder may be rash.

In another aspect, the present application provides a use of the compound as shown in Formula I or a pharmaceutically acceptable salt, prodrug, isotopic variant, or solvate thereof in preparing a medicament, the medicament is used for preventing, relieving and/or treating EGFR inhibitor-associated skin disease or disorder in a subject. For example, the skin disease or disorder may be rash. For example, the compound as shown in Formula I may be

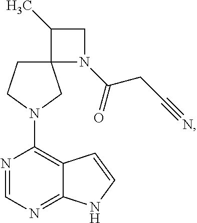 [SMILES: CC1CN(C(=O)CC#N)C12CCN(c1ncnc3[nH]ccc13)C2]

In another aspect, the present application provides a use of the compound as shown in Formula I or a pharmaceutically acceptable salt, prodrug, isotopic variant, or solvate thereof in preparing a medicament, the medicament is used for preventing, relieving and/or treating MEK inhibitor-associated skin disease or disorder in a subject. For example, the skin disease or disorder max be rash. For example, the compound as shown in Formula I may be

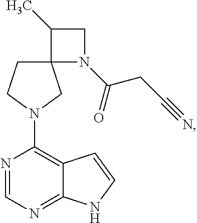 [SMILES: CC1CN(C(=O)CC#N)C12CCN(c1ncnc3[nH]ccc13)C2]

In another aspect the present application provides a use of the compound as shown in Formula I or a pharmaceutically acceptable salt, prodrug, isotopic variant, or solvate thereof in preparing a medicament, the medicament is used for preventing, relieving and/or treating VEGFR inhibitor-associated skin disease or disorder in a subject. For example, the skin disease or disorder may be rash. For example, the compound as shown in Formula I may be

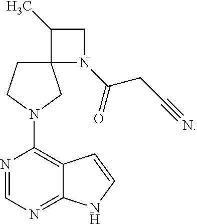 [SMILES: CC1CN(C(=O)CC#N)C12CCN(c1ncnc3[nH]ccc13)C2]

In mother aspect, the present application provides a use of the compound as shown in Formula I or a pharmaceutically acceptable salt, prodrug, isotopic variant, or solvate thereof in preparing a medicament, the medicament is Used for preventing, relieving and/or treating PI3K inhibitor-associated skin disease or disorder in a subject. For example, the skin disease or disorder may be rash. For example, the compound as shown in Formula I may be

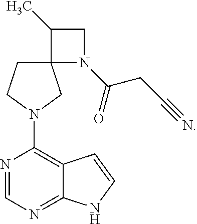 [SMILES: CC1CN(C(=O)CC#N)C12CCN(c1ncnc3[nH]ccc13)C2]

In another aspect, the present application provides a use of the compound as shown in Formula I or a pharmaceutically acceptable salt, prodrug, isotopic variant, or solvate thereof in preparing a medicament, the medicament is used for preventing, relieving and/or treating immune checkpoint inhibitor-associated skin disease or disorder in a subject. For example, the skin disease or disorder may be rash. For example, the compound as shown in Formula I may be

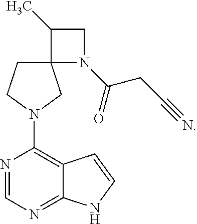 [SMILES: CC1CN(C(=O)CC#N)C12CCN(c1ncnc3[nH]ccc13)C2]

In another aspect, the present application provides a method, comprising the following steps:
1) monitoring skin diseases or disorders of a subject who has been administered with an antitumor agent;
2) when the monitoring shows that the subject develops skin diseases or disorders associated with administration of the antitumor agent, administering the compound as shown in Formula I of the present application or a pharmaceutically acceptable salt, prodrug, isotopic variant, or solvate thereof to the subject.

In some embodiments, the compound as shown in Formula I

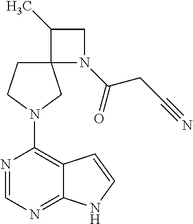 [SMILES: CC1CN(C(=O)CC#N)C12CCN(c1ncnc3[nH]ccc13)C2]

or a pharmaceutically acceptable salt, prodrug, isotopic variant, or solvate thereof (for example) may be administered locally and/or transdermally. In some embodiments, the concentration of the compound as shown in Formula I (e.g.,

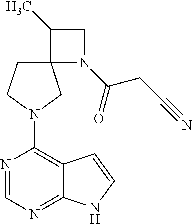 [SMILES: CC1CN(C(=O)CC#N)C12CCN(c1ncnc3[nH]ccc13)C2]

or a pharmaceutically acceptable salt, prodrug, isotopic variant, or solvate thereof) may be about 0.01%-about 5%. In some embodiments, the administration frequency of the compound as shown in Formula I

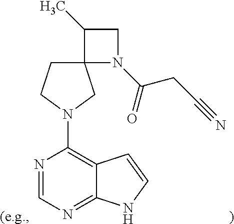

or a pharmaceutically acceptable salt, prodrug, isotopic variant, or solvate thereof may be once a day, twice a day or 3 times a day. In some embodiments, the administration cycle of the compound as shown in Formula I

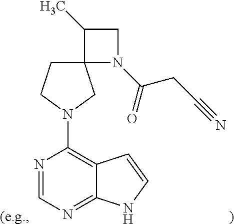

or a pharmaceutically acceptable salt, prodrug, isotopic variant, or solvate thereof may be 1-week to 3 weeks.

In some embodiments, the compound as shown in Formula I

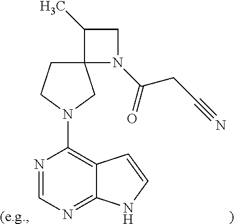

or a pharmaceutically acceptable salt, prodrug, isotopic variant, or solvate thereof may be administered locally and/or transdermally, the concentration may be about 0.01%-about 5%, and the administration frequency may be once a day, twice a day or 3 times a day (e.g., once a day), and the administration cycle may be 1 week to 3 weeks.

In the present application, the compound as shown in Formula I

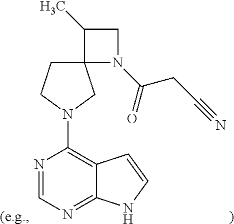

or a pharmaceutically acceptable salt, prodrug, isotopic variant, or solvate thereof can be used for treating a skin disease or disorder (e.g., rash) associated with an immunotherapeutic agent (e.g., PD-1 antibody, PD-L1 antibody and/or CTLA-4 antibody), and the compound as shown in Formula I

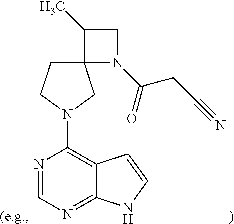

or a pharmaceutically acceptable salt, prodrug, isotopic variant, or solvate thereof may be administered locally and/or transdermally. For example, the concentration may be about 0.01%-about 5%.

For example, the compound

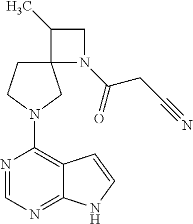 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated a PD-1/PD-L1 inhibitor (e.g., Pembrolizumab and/or Nivolumab), and the compound

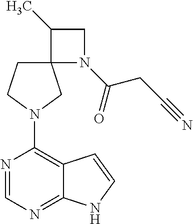 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For sample, the concentration of the compound may be about 0.5%.

For example, the compound

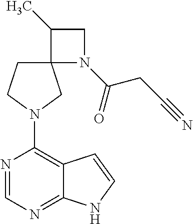 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated With a CTLA-4 inhibitor (e.g., Ipilimumab), and the compound

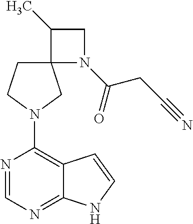 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.5%.

For example, the compound

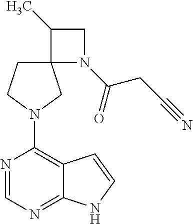 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with the combination of a CTLA-4 inhibitor and a PD-1/PD-L1 inhibitor (e.g., the combination of Ipilimumab and Nivolumab), and the compound

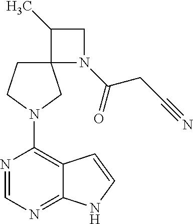 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.5%.

In the present application, the compound as shown in Formula I

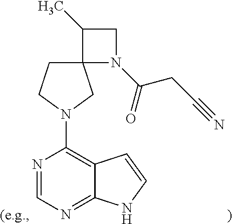 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with a targeted therapy inhibitor (e.g., tyrosinase inhibitor), and the compound as shown in Formula I

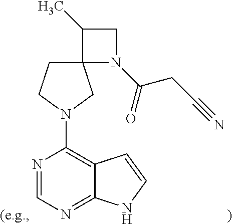 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5%.

In the present application, the compound as shown in Formula I

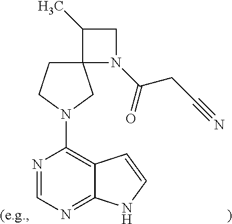 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with an EGFR inhibitor (including a small molecular compound and/or an anti-EGFR antibody), and the compound as shown in Formula I

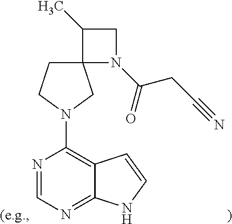 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5%.

For example, the compound

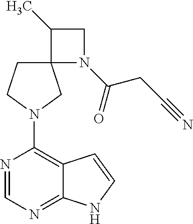 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with afatinib, and the compound

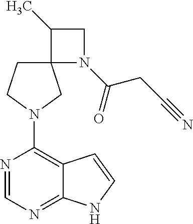 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%.

For example, the compound

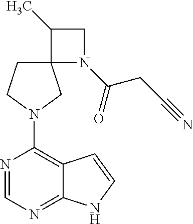 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with erlotinib, and the compound

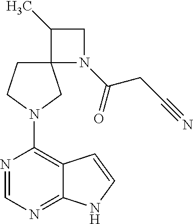 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.5%-about 2%.

For example, the compound

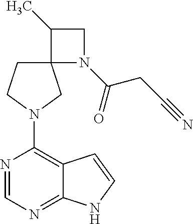 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with osimertinib, and the compound

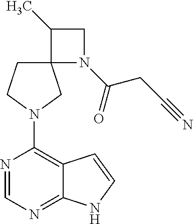 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%-about 5%.

For example, the compound

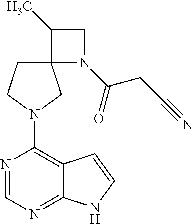 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with cetuximab, and the compound

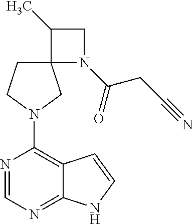 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%-about 5%.

For example, the compound

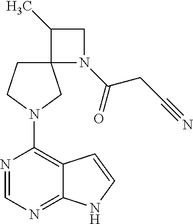 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with panitumumab, and the compound

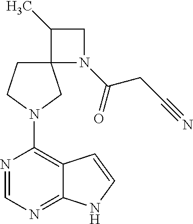 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%-about 1.5%.

In the present application, the compound as shown in Formula I

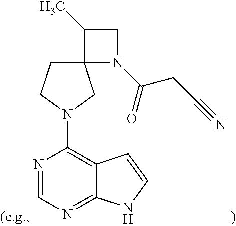 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with VEGFR inhibitor, and the compound as shown in Formula I

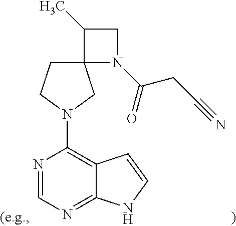 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.05%-about 2.0%).

For example, the compound

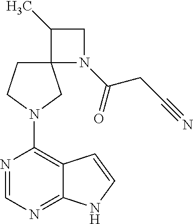 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with sorafenib, and the compound

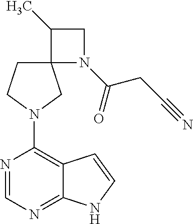 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%.

For example, the compound

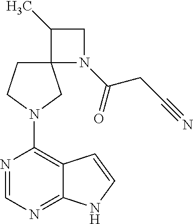 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with apatinib, and the compound

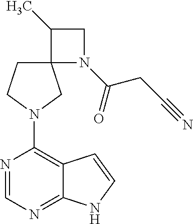 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%.

In the present application, the compound as shown in Formula I

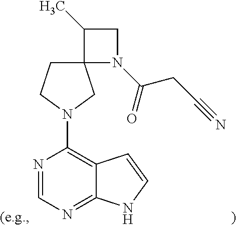 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with MEK inhibitor, and the compound as shown in Formula I

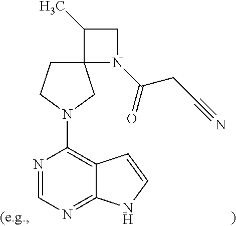 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.5%-about 2.0%).

In the present application, the compound as shown in Formula I

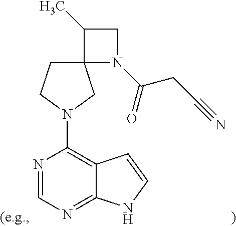 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with ALK inhibitor, and the compound as shown in Formula I

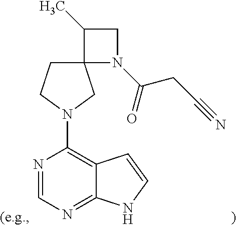 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.5%-about 1.5%).

In the present application, the compound as shown in Formula I

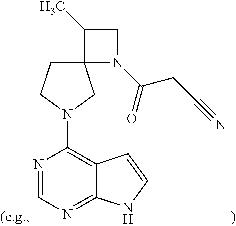 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with FGFR inhibitor, and the compound as shown in Formula I

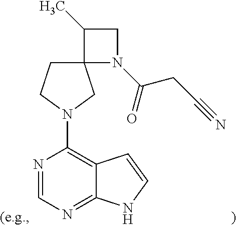 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5%.

In the present application, the compound as shown in Formula I

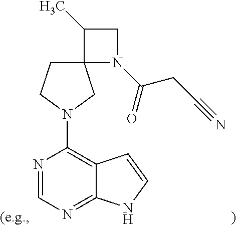 [SMILES: CC1CN(C(=O)CC#N)C12CCN(c1ncnc3[nH]ccc13)C2]

or a pharmaceutical) acceptable salt, prodrug, isotopic variant, or solvate thereof can be used for treating a skin disease or disorder (e.g., rash) associated With mTOR (e.g., mTORC1 and/or mTORC2) inhibitor, and the compound as shown in Formula I

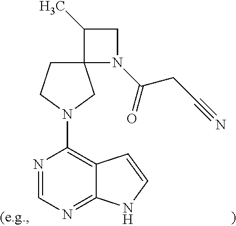 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5%.

In the present application, the compound as shown in Formula I

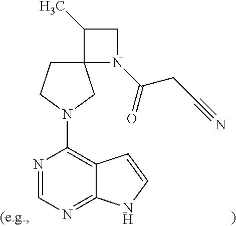 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with BTK inhibitor, and the compound as shown in Formula I

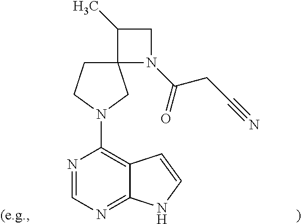 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.5%-about 5.0%).

In the present application, the compound as shown in Formula I

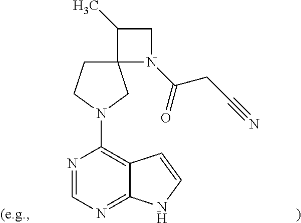 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with PI3K inhibitor, and the compound as shown in Formula I

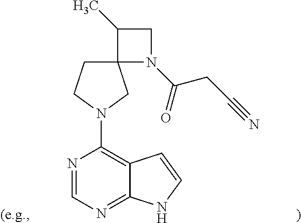 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.5%-about 5.0%).

In the present application, the compound as shown in Formula I

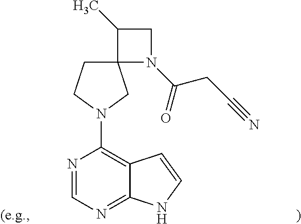 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with FAK inhibitor, and the compound as shown in Formula I

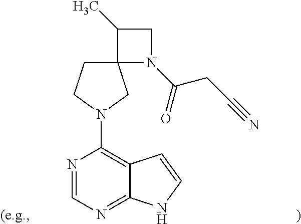 [SMILES: CC1CN(C(=O)CC#N)C12CCN(c1ncnc3[nH]ccc13)C2]

a pharmaceutically acceptable salt, prodrug, isotopic variant, or solvate thereof may be administered locally and/or transdermally. For example, the concentration may be about 0.05%-about 5% (e.g., about 0.1%-about 5.0%).

For example, the compound

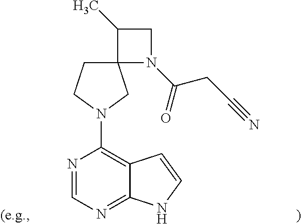 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with defactinib, and the compound

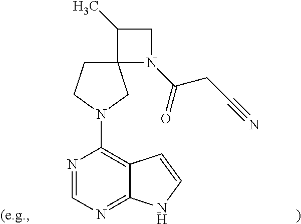 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.1%-about 5.0%.

In the present application, the compound as shown in Formula I

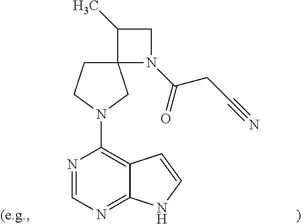 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with EGFR/Met double-target inhibitor, and the compound as shown in Formula I

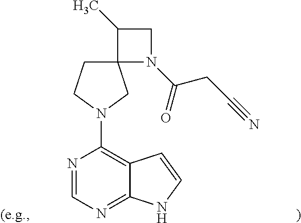 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration may be about 0.05%-about 5% (e.g., about 0.05%-about 1.5%).

For example, the compound

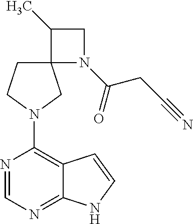 [SMILES: CC1CN(C(=O)CC#N)C12CCN(c1ncnc3[nH]ccc13)C2]

or a pharmaceutically acceptable salt, prodrug, isotopic variant, or solvate thereof can be used for treating a skin disease or disorder (e.g., rash) associated with amivantamab, and the compound

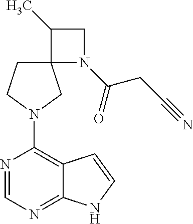 [SMILES: CC1CN(C(=O)CC#N)C12CCN(c1ncnc3[nH]ccc13)C2]

or a pharmaceutically acceptable salt, prodrug, isotopic variant, or solvate thereof may be administered locally and/or transdermally. For example, the concentration of the compound may be about 0.01%-about 5%. For example, the concentration of the compound may be about 0.05%-about 1.5%.

Without being limited by any theory, the following examples are only for the purpose of illustrating various technical schemes of the present application, and not intended to limit the invention scope of the present application.

### EXAMPLES

### Examples 1-96: Experiment for Demonstrating the Ability of Delgocitinib in Preventing the Occurrence of Rashes Caused by a Small Molecular Antitumor Agent in Rat Models

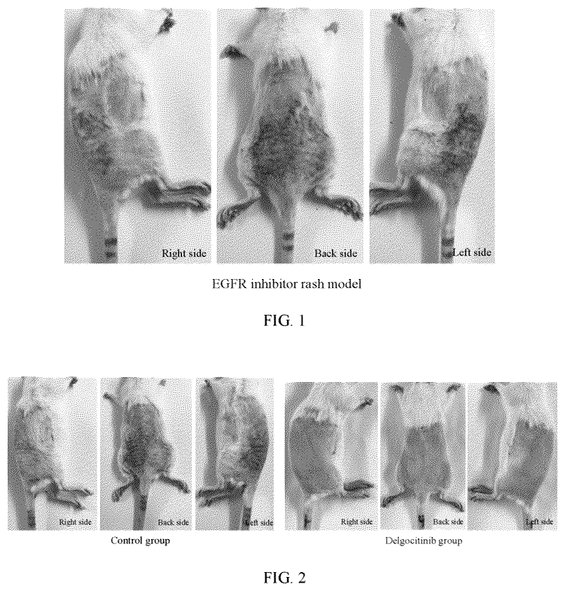

Construction of rat rash models: An antitumor agent was daily administered to 6-week female SD rats, and after several days, a large area of rashes appeared on the back of the rats (the photographs are shown in FIG. 1). There was no difference between the left and right sides of the rash area, and the rash grade was similar on both sides, which were like the conditions in human. Thus, rats are very good animal models to mimic the rashes caused by the antitumor agent.

After a week of acclimatization (about 200 g), the SD rats were divided into 10 rats per group. The hair on the back of the rats was shaved with an electric shaver on the day before the experiment, and then the administration experiment was performed. The antitumor agent was dissolved in a suitable solvent for gavage/injection administration to all the rats, for which the administration mode, dosage and frequency were shown in Table 1. The experiment was divided into a delgocitinib group and a control group. After administration of the antitumor agent to rats, the back of the rats in the delgocitinib group (about 3 cm×3 cm) was applied with delgocitinib ointment (the concentration thereof was shown in Table 1, about 0.5 g); the back of the rats in the control group (about 3 cm×3 cm) was applied with blank ointment (about 0.5 g). After applying the medicament, the rats were fixed by a cylinder for about 4 hrs. Then, the rats were released, wiped with clean water to remove the residual medicament at the application site, and returned to the cage for normal breeding. Delgocitinib and the blank ointment were applied once a day. The experiments of administration of the antitumor agent and application of the ointment were repeated until the rats in the control group developed apparent rashes. At this point, the number of rats in the delgocitinib group on which the skin kept normal or remarkably milder than the rashes of the rats in the control group was calculated as the number of rats on which rashes had been effectively inhibited.

Table 1 lists various animal experiment combinations of antitumor agents and delgocitinib ointment, as well as the corresponding experimental results (wherein, the numerical values in the column of relative remission rate=the number of rats in the delgocitinib group on which rashes were milder than those of rats in the control group/the total number of rats in the delgocitinib group×100%).

TABLE 1
Experimental conditions and results of Examples 1-96

| | Small | | | | | | | | Relative |
|---|---|---|---|---|---|---|---|---|---|
| | molecular | | | | | | | | remission |
| Example | antitumor | | Dosage | Administration | | Medicament | Concentration | | rate |
| No. | agent | Target | (mg/kg) | mode | Frequency | applied | wt % | Days | (%) |
| 1 | Afatinib | EGFR | 40 | Gavage | Once a | Delgocitinib | 0.05% | 14 | 70% |
| | | | | | day | ointment | | | |
| 2 | Afatinib | EGFR | 40 | Gavage | Once a | Delgocitinib | 0.10% | 14 | 90% |
| | | | | | day | ointment | | | |
| 3 | Afatinib | EGFR | 40 | Gavage | Once a | Delgocitinib | 0.30% | 14 | 100% |
| | | | | | day | ointment | | | |
| 4 | Afatinib | EGFR | 40 | Gavage | Once a | Delgocitinib | 0.50% | 14 | 100% |
| | | | | | day | ointment | | | |
| 5 | Afatinib | EGFR | 40 | Gavage | Once a | Delgocitinib | 0.80% | 14 | 100% |
| | | | | | day | ointment | | | |
| 6 | Afatinib | EGFR | 40 | Gavage | Once a | Delgocitinib | 1.50% | 14 | 100% |
| | | | | | day | ointment | | | |
| 7 | Afatinib | EGFR | 40 | Gavage | Once a | Delgocitinib | 2.00% | 14 | 100% |
| | | | | | day | ointment | | | |
| 8 | Afatinib | EGFR | 40 | Gavage | Once a | Delgocitinib | 5% | 14 | 100% |
| | | | | | day | ointment | | | |
| 9 | Dacomitinib | EGFR | 10 | Gavage | Once a | Delgocitinib | 0.05% | 12 | 70% |
| | | | | | day | ointment | | | |
| 10 | Dacomitinib | EGFR | 10 | Gavage | Once a | Delgocitinib | 0.50% | 12 | 100% |
| | | | | | day | ointment | | | |
| 11 | Dacomitinib | EGFR | 10 | Gavage | Once a | Delgocitinib | 1.50% | 12 | 80% |
| | | | | | day | ointment | | | |
| 12 | Dacomitinib | EGFR | 10 | Gavage | Once a | Delgocitinib | 2% | 12 | 100% |
| | | | | | day | ointment | | | |
| 13 | Dacomitinib | EGFR | 10 | Gavage | Once a | Delgocitinib | 5% | 12 | 100% |
| | | | | | day | ointment | | | |
| 14 | Osimertinib | EGFR | 60 | Gavage | Twice a | Delgocitinib | 0.05% | 10 | 80% |
| | | | | | day | ointment | | | |
| 15 | Osimertinib | EGFR | 60 | Gavage | Twice a | Delgocitinib | 0.50% | 10 | 100% |
| | | | | | day | ointment | | | |
| 16 | Osimertinib | EGFR | 60 | Gavage | Twice a | Delgocitinib | 2% | 10 | 100% |
| | | | | | day | ointment | | | |
| 17 | Osimertinib | EGFR | 60 | Gavage | Twice a | Delgocitinib | 5% | 10 | 100% |
| | | | | | day | ointment | | | |
| 18 | EAI045 | EGFR | 80 | Gavage | Once a | Delgocitinib | 0.05% | 11 | 70% |
| | | | | | day | ointment | | | |
| 19 | EAI045 | EGFR | 80 | Gavage | Once a | Delgocitinib | 0.50% | 11 | 100% |
| | | | | | day | ointment | | | |
| 20 | EAI045 | EGFR | 80 | Gavage | Once a | Delgocitinib | 2% | 11 | 100% |
| | | | | | day | ointment | | | |
| 21 | EAI045 | EGFR | 80 | Gavage | Once a | Delgocitinib | 5% | 11 | 100% |
| | | | | | day | ointment | | | |
| 22 | Gefitinib | EGFR | 80 | Gavage | Twice a | Delgocitinib | 0.05% | 15 | 60% |
| | | | | | day | ointment | | | |
| 23 | Gefitinib | EGFR | 80 | Gavage | Twice a | Delgocitinib | 0.50% | 15 | 80% |
| | | | | | day | ointment | | | |
| 24 | Gefitinib | EGFR | 80 | Gavage | Twice a | Delgocitinib | 1.50% | 15 | 100% |
| | | | | | day | ointment | | | |
| 25 | Gefitinib | EGFR | 80 | Gavage | Twice a | Delgocitinib | 2% | 15 | 100% |
| | | | | | day | ointment | | | |
| 26 | Gefitinib | EGFR | 80 | Gavage | Twice a | Delgocitinib | 5% | 15 | 100% |
| | | | | | day | ointment | | | |
| 27 | Almonertinib | EGFR | 80 | Gavage | Once a | Delgocitinib | 0.50% | 12 | 80% |
| | | | | | day | ointment | | | |
| 28 | Pyrotinib | EGFR | 50 | Gavage | Once a | Delgocitinib | 0.50% | 15 | 90% |
| | | | | | day | ointment | | | |
| 29 | Brigatinib | ALK/EGFR | 40 | Gavage | Once a | Delgocitinib | 0.50% | 13 | 100% |
| | | | | | day | ointment | | | |
| 30 | Neratinib | EGFR | 60 | Gavage | Once a | Delgocitinib | 0.50% | 10 | 60% |
| | | | | | day | ointment | | | |
| 31 | Olmutinib | EGFR | 80 | Gavage | Once a | Delgocitinib | 0.50% | 10 | 100% |
| | | | | | day | ointment | | | |
| 32 | Bosutinib | BCR/ABL | 30 | Gavage | Once a | Delgocitinib | 0.50% | 12 | 60% |
| | | | | | day | ointment | | | |
| 33 | Icotinib | EGFR | 60 | Gavage | Once a | Delgocitinib | 0.50% | 15 | 60% |
| | | | | | day | ointment | | | |
| 34 | Vandetanib | VEGFR, | 40 | Gavage | Once a | Delgocitinib | 0.50% | 15 | 80% |
| | | EGFR, | | | day | ointment | | | |
| | | ABL | | | | | | | |
| 35 | Lapatinib | HER1, | 120 | Gavage | Once a | Delgocitinib | 0.50% | 10 | 70% |
| | | HER2 | | | day | ointment | | | |
| 36 | Alflutinib | EGFR | 30 | Gavage | Once a | Delgocitinib | 2% | 16 | 60% |
| | | | | | day | ointment | | | |
| 37 | BPI-7711 | EGFR | 120 | Gavage | Once a | Delgocitinib | 2% | 10 | 90% |
| | | | | | day | ointment | | | |
| 38 | Mobocertinib | EGFR, | 20 | Gavage | Once a | Delgocitinib | 2% | 15 | 80% |
| | | HER2 | | | day | ointment | | | |
| 39 | Dovitinib | EGFR, | 60 | Gavage | Once a | Delgocitinib | 2% | 13 | 100% |
| | | FGFR1/3, | | | day | ointment | | | |
| | | VEGFR/2 | | | | | | | |
| 40 | Zorifertinib | EGFR | 40 | Gavage | Once a | Delgocitinib | 2% | 15 | 100% |
| | | | | | day | ointment | | | |
| 41 | Varlitinib | EGFR | 80 | Gavage | Once a | Delgocitinib | 0.50% | 10 | 60% |
| | | | | | day | ointment | | | |
| 42 | Orelabrutinib | BTK | 20 | Gavage | Once a | Delgocitinib | 0.50% | 10 | 50% |
| | | | | | day | ointment | | | |
| 43 | Tirabrutinib | BTK | 40 | Gavage | Once a | Delgocitinib | 0.50% | 10 | 60% |
| | | | | | day | ointment | | | |
| 44 | Zanubrutinib | BTK | 25 | Gavage | Twice a | Delgocitinib | 1.50% | 10 | 100% |
| | | | | | day | ointment | | | |
| 45 | Acalabrutinib | BTK | 30 | Gavage | Once a | Delgocitinib | 0.50% | 10 | 80% |
| | | | | | day | ointment | | | |
| 46 | Ibrutinib | BTK | 30 | Gavage | Once a | Delgocitinib | 0.50% | 10 | 60% |
| | | | | | day | ointment | | | |
| 47 | Dasatinib | Bcr-Abl1, | 100 | Gavage | Once a | Delgocitinib | 1.50% | 10 | 100% |
| | | BTK | | | day | ointment | | | |
| 48 | Pirtobrutinib | BTK | 35 | Gavage | Twice a | Delgocitinib | 0.50% | 10 | 70% |
| | | | | | day | ointment | | | |
| 49 | Tolebrutinib | BTK | 30 | Gavage | Once a | Delgocitinib | 2% | 10 | 100% |
| | | | | | day | ointment | | | |
| 50 | Rilzabrutinib | BTK | 40 | Gavage | Twice | Delgocitinib | 2% | 10 | 100% |
| | | | | | day | ointment | | | |
| 51 | Fenebrutinib | BTK | 40 | Gavage | Once a | Delgocitinib | 5% | 10 | 100% |
| | | | | | day | ointment | | | |
| 52 | Evobrutinib | BTK | 40 | Gavage | Once a | Delgocitinib | 5% | 10 | 100% |
| | | | | | day | ointment | | | |
| 53 | Selumetinib | MEK | 100 | Gavage | Once a | Delgocitinib | 0.50% | 10 | 80% |
| | | | | | day | ointment | | | |
| 54 | Binimetinib | MEK | 50 | Gavage | Once a | Delgocitinib | 0.50% | 10 | 100% |
| | | | | | day | ointment | | | |
| 55 | Cobimetinib | MEK | 25 | Gavage | Once a | Delgocitinib | 0.50% | 15 | 60% |
| | | | | | day | ointment | | | |
| 56 | Trametinib | MEK | 20 | Tail vein | 3 times a | Delgocitinib | 0.50% | 10 | 60% |
| | | | | injection | week | ointment | | | |
| 57 | Regorafenib | VEGFR, | 300 | Gavage | Once a | Delgocitinib | 0.50% | 12 | 70% |
| | | FGFR, | | | day | ointment | | | |
| | | c-KIT | | | | | | | |
| 58 | GSK- | MEK | 10 | Gavage | Once a | Delgocitinib | 0.50% | 16 | 100% |
| | 1120212 | | | | day | ointment | | | |
| 59 | Alpelisib | PI3K | 65 | Gavage | Once a | Delgocitinib | 0.50% | 10 | 80% |
| | | | | | day | ointment | | | |
| 60 | Duvelisib | PI3K | 60 | Gavage | Twice a | Delgocitinib | 0.50% | 10 | 80% |
| | | | | | day | ointment | | | |
| 61 | Copanlisib | PI3K | 20 | Intraperitoneal | Once a | Delgocitinib | 0.50% | 15 | 70% |
| | | | | injection | day | ointment | | | |
| 62 | Idelalisib | PI3K, | 60 | Gavage | Twice a | Delgocitinib | 2.00% | 16 | 100% |
| | | BCRP | | | day | ointment | | | |
| 63 | Nortriptyline | PI3K | 60 | Intraperitoneal | Once a | Delgocitinib | 2.00% | 10 | 100% |
| | | | | injection | day | ointment | | | |
| 64 | Inavolisib | PI3K | 10 | Gavage | Once a | Delgocitinib | 2.00% | 14 | 60% |
| | | | | | day | ointment | | | |
| 65 | Dactolisib | PI3K | 60 | Gavage | Once a | Delgocitinib | 2.00% | 15 | 100% |
| | | | | | day | ointment | | | |
| 66 | Apitolisib | PI3K | 30 | Gavage | Once a | Delgocitinib | 0.50% | 15 | 50% |
| | | | | | day | ointment | | | |
| 67 | Parsaclisib | PI3K | 100 | Gavage | Once a | Delgocitinib | 0.50% | 10 | 60% |
| | | | | | day | ointment | | | |
| 68 | Buparlisib | PI3K | 40 | Gavage | Once a | Delgocitinib | 1.50% | 15 | 100% |
| | | | | | day | ointment | | | |
| 69 | Rigosertib | PI3K | 100 | Intraperitoneal | Once a | Delgocitinib | 1.50% | 9 | 80% |
| | | | | injection | day | ointment | | | |
| 70 | Enzastaurin | PI3K | 130 | Gavage | Twice a | Delgocitinib | 5.00% | 10 | 100% |
| | | | | | day | ointment | | | |
| 71 | Paxalisib | PI3K | 40 | Gavage | Once a | Delgocitinib | 5.00% | 15 | 100% |
| | | | | | day | ointment | | | |
| 72 | Leniolisib | PI3K | 70 | Gavage | Twice a | Delgocitinib | 5.00% | 10 | 100% |
| | | | | | day | ointment | | | |
| 73 | Ipatasertib | AKT | 120 | Gavage | Once a | Delgocitinib | 0.50% | 10 | 100% |
| | | | | | day | ointment | | | |
| 74 | Zotarolimus | mTORC1, | 4(iv) | Tail vein | Once a | Delgocitinib | 0.05% | 20 | 40% |
| | | mTORC2 | | injection | day | ointment | | | |
| 75 | Sirolimus | mTORC1, | 15 | Gavage | Once a | Delgocitinib | 0.50% | 10 | 60% |
| | | mTORC2 | | | day | ointment | | | |
| 76 | Everolimus | mTORC1, | 10 | Gavage | Once a | Delgocitinib | 2.00% | 14 | 100% |
| | | mTORC2 | | | day | ointment | | | |
| 77 | Temsirolimus | mTORC1, | 50 | Tail vein | Once a | Delgocitinib | 5.00% | 10 | 100% |
| | | mTORC2 | | injection | day | ointment | | | |
| 78 | Sorafenib | BRAF, | 60 | Gavage | Twice | Delgocitinib | 0.05% | 10 | 40% |
| | | VEGFR | | | day | ointment | | | |
| 79 | Apatinib | VEGFR | 90 | Gavage | Once a | Delgocitinib | 0.05% | 12 | 50% |
| | | | | | day | ointment | | | |
| 80 | Lenvatinib | VEGFR | 65 | Gavage | Once a | Delgocitinib | 0.50% | 16 | 50% |
| | | | | | day | ointment | | | |
| 81 | Regorafenib | BRAF, | 90 | Gavage | Once a | Delgocitinib | 0.50% | 15 | 40% |
| | | VEGFR | | | day | ointment | | | |
| 82 | Sunitinib | VEGFR | 120 | Gavage | Once a | Delgocitinib | 1.00% | 14 | 70% |
| | | | | | day | ointment | | | |
| 83 | Cabozantinib | VEGFR | 50 | Gavage | Once a | Delgocitinib | 1.00% | 14 | 70% |
| | | | | | day | ointment | | | |
| 84 | Axitinib | VEGFR | 50 | Gavage | Once a | Delgocitinib | 1.50% | 16 | 70% |
| | | | | | day | ointment | | | |
| 85 | Nintedanib | VEGFR | 110 | Gavage | Once a | Delgocitinib | 1.50% | 14 | 90% |
| | | | | | day | ointment | | | |
| 86 | Brivanib | VEGFR | 80 | Gavage | Once a | Delgocitinib | 1.50% | 16 | 100% |
| | | | | | day | ointment | | | |
| 87 | Vatalanib | VEGFR | 150 | Gavage | Once a | Delgocitinib | 2.00% | 10 | 100% |
| | | | | | day | ointment | | | |
| 88 | Fruquintinib | VEGFR | 40 | Gavage | Once a | Delgocitinib | 2.00% | 16 | 100% |
| | | | | | day | ointment | | | |
| 89 | Dabrafenib | BRAF | 100 | Gavage | Once a | Delgocitinib | 2.00% | 14 | 100% |
| | | | | | day | ointment | | | |
| 90 | Vemurafenib | BRAF | 110 | Gavage | Twice a | Delgocitinib | 2.00% | 12 | 100% |
| | | | | | day | ointment | | | |
| 91 | Encorafenib | BRAF | 70 | Gavage | Once a | Delgocitinib | 5.00% | 16 | 100% |
| | | | | | day | ointment | | | |
| 92 | Pazopanib | BRAF | 100 | Gavage | Once a | Delgocitinib | 5.00% | 18 | 100% |
| | | | | | day | ointment | | | |
| 93 | Crizotinib | ALK | 50 | Gavage | Once a | Delgocitinib | 1.50% | 15 | 40% |
| | | | | | day | ointment | | | |
| 94 | Panobinostat | HDAC | 20 | Intraperitoneal | Once a | Delgocitinib | 2% | 11 | 50% |
| | | | | injection | day | ointment | | | |
| 95 | Defactinib | FAK | 60 | Gavage | Once a | Delgocitinib | 0.1% | 15 | 60% |
| | | | | | day | ointment | | | |
| 96 | Defactinib | FAK | 60 | Gavage | Once a | Delgocitinib | 5% | 15 | 100% |
| | | | | | day | ointment | | | |

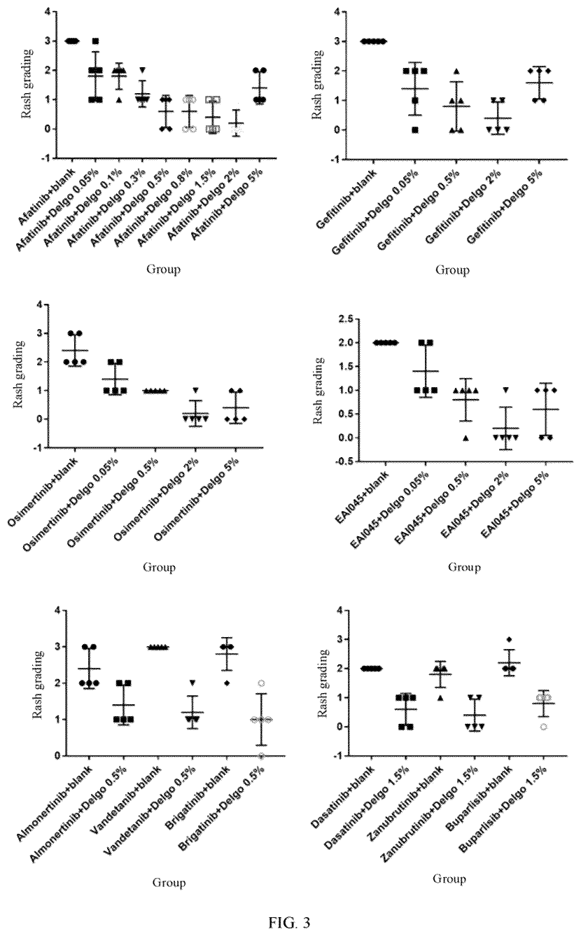

FIG. 2 shows the photographs of left side, back side, and right side of typical rats in the control group and the delgocitinib group in Table 1. FIG. 3 shows the partial rash grading results of the delgocitinib group and the control group at the end of the experiment.

It can be seen from the results in Table 1 and FIGS. 2-3 that: the delgocitinib ointment can effectively prevent rashes caused by a small molecular antitumor agent.

### Examples 97-108: Experiment for Demonstrating the Ability of Delgocitinib in Preventing the Occurrence of Rashes Caused by Antibody-Type Antitumor Agents in Rat Models

After a week of acclimatization (about 200 g), the SD rats were divided into 10 rats per group. The hair on the back of the rats was shaved with an electric shaver on the day before the experiment, and then the administration experiment was performed. The experiment was divided into a delgocitinib group and a control group. The administration frequency, dosage and injection rate of the antibody-type antitumor agents were shown in Table 2. During the experiment, the back ofthe rats in the delgocitinib group (about 3 cm×3 cm) was applied with delgocitinib ointment; the back of the rats in the control group (about 3 cm×3 cm) was applied with blank ointment (about 0.5 g). After applying the medicament, the rats were fixed by a cylinder for about 4 hrs. Then, the rats were released, wiped with clean water to remove the residual medicament at the application site, and returned to the cage for normal breeding. The ointment was applied once a day. The experiment ended when the rats in the control group developed apparent rashes. The number of rats in the delgocitinib group on which the skin kept normal or remarkably milder than the rashes of the rats in the control group at the end ofthe experiment was calculated as the number of rats on which rashes had been effectively inhibited.

Table 2 lists various animal experiment combinations of antibody-type antitumor agents and delgocitinib ointment, as well as the corresponding experimental results (wherein, the numerical values in the column of relative remission rate=the number of rats in the delgocitinib group on which rashes were milder than those of rats in the control group/the total number of rats in the delgocitinib group×100%).

TABLE 2
Experimental conditions and results of examples 97-108

| | Antibody- | | | | | | | | Relative |
|---|---|---|---|---|---|---|---|---|---|
| | type | | | | | | | | remission |
| Example | antitumor | | | Administration | | Medicament | Concentration | | rate |
| No. | agent | Target | Dosage | mode | Frequency | applied | wt % | Days | (%) |
| 97 | Cetuximab | EGFR | 100 mg/kg, 15 | Tail | Twice a | Delgocitinib | 0.05% | 10 | 50% |
| | | | min | vein | week | ointment | | | |
| | | | with an injection | injection | | | | | |
| | | | rate of 1.3 | | | | | | |
| | | | ml/kg/min for | | | | | | |
| | | | each time | | | | | | |
| 98 | Cetuximab | EGFR | 100 mg/kg, 15 | Tail | Twice a | Delgocitinib | 0.50% | 10 | 70% |
| | | | min | vein | week | ointment | | | |
| | | | with an injection | injection | | | | | |
| | | | rate of 1.3 | | | | | | |
| | | | ml/kg/min for | | | | | | |
| | | | each time | | | | | | |
| 99 | Cetuximab | EGFR | 100 mg/kg, 15 | Tail | Twice a | Delgocitinib | 1.50% | 10 | 80% |
| | | | min | vein | week | ointment | | | |
| | | | with an injection | injection | | | | | |
| | | | rate of 1.3 | | | | | | |
| | | | ml/kg/min for | | | | | | |
| | | | each time | | | | | | |
| 100 | Cetuximab | EGFR | 100 mg/kg, 15 | Tail | Twice a | Delgocitinib | 2.00% | 10 | 100% |
| | | | min | vein | week | ointment | | | |
| | | | with an injection | injection | | | | | |
| | | | rate of 1.3 | | | | | | |
| | | | ml/kg/min for | | | | | | |
| | | | each time | | | | | | |
| 101 | Panitumumab | EGFR | 60 | Tail | Once a | Delgocitinib | 0.05% | 10 | 40% |
| | | | | vein | week | ointment | | | |
| | | | with an injection | injection | | Delgocitinib | 1.50% | 10 | 100% |
| | | | rate of 1.3 | | | ointment | | | |
| | | | ml/kg/min for | | | | | | |
| | | | each time | | | | | | |
| 102 | Rituximab | CD20 | 60 | Tail | Once a | Delgocitinib | 0.50% | 10 | 60% |
| | | | | vein | week | ointment | | | |
| | | | with an injection | injection | | Delgocitinib | 1.50% | 10 | 100% |
| | | | rate of 1.3 | | | ointment | | | |
| | | | ml/kg/min for | | | | | | |
| | | | each time | | | | | | |
| 103 | Amivantaab | EGFR/ | 30 | Tail | Twice a | Delgocitinib | 0.50% | 10 | 70% |
| | | c-Met | with an injection | vein | week | ointment | | | |
| | | | rate of 1.3 | injection | | | | | |
| | | | ml/kg/min for | | | | | | |
| | | | each time | | | | | | |
| 104 | Amivantamab | EGFR/ | 30 | Tail | Twice a | Delgocitinib | 1.50% | 10 | 90% |
| | | c-Met | with an injection | vein | week | ointment | | | |
| | | | rate of 1.3 | injection | | | | | |
| | | | ml/kg/min for | | | | | | |
| | | | each time | | | | | | |
| 105 | LY3164530 | EGFR/ | 15 | Tail | Once a | Delgocitinib | 0.50% | 10 | 50% |
| | | c-Met | with an injection | vein | week | ointment | | | |
| | | | rate | injection | | | | | |
| | | | of 1.3 | | | | | | |
| | | | ml/kg/min for | | | | | | |
| | | | each time | | | | | | |
| 106 | LY3164530 | EGFR/ | 15 | Tail | Once a | Delgocitinib | 1.50% | 10 | 100% |
| | | c-Met | with an injection | vein | week | ointment | | | |
| | | | rate of 1.3 | injection | | | | | |
| | | | ml/kg/min for | | | | | | |
| | | | each time | | | | | | |
| 107 | EMB-01 | EGFR/ | 10 | Tail | Twice a | Delgocitinib | 0.50% | 10 | 60% |
| | | c-Met | with an injection | vein | week | ointment | | | |
| | | | rate of 1.3 | injection | | | | | |
| | | | ml/kg/min for | | | | | | |
| | | | each time | | | | | | |
| 108 | EMB-01 | EGFR/ | 10 | Tail | Twice a | Delgocitinib | 1.50% | 10 | 100% |
| | | c-Met | with an injection | vein | week | ointment | | | |
| | | | rate of 1.3 | injection | | | | | |
| | | | ml/kg/min for | | | | | | |
| | | | each time | | | | | | |

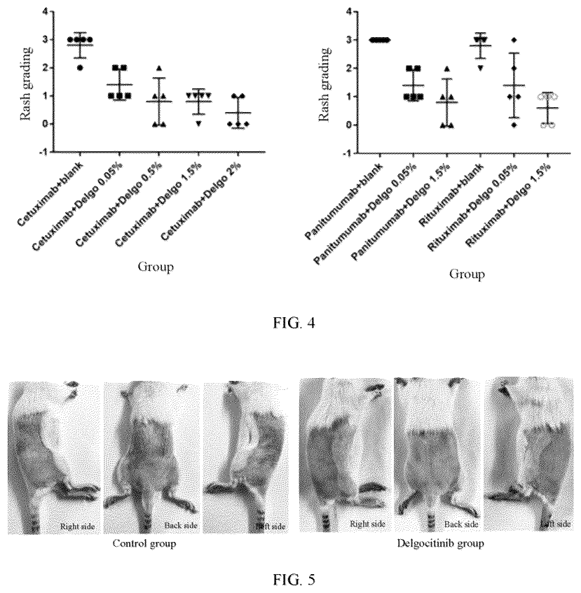

FIG. 4 shows the partial rash grading results of the delgocitinib group and the control group at the end of the experiment.

It can be seen from the results in Table 2 and FIG. 4 that: Delgocitinib ointment can effectively prevent rashes caused by antibody-type antitumor agents.

### Examples 109-136: Experiment for Demonstrating the Ability of Delgocitinib in Treating Rashes Caused by a Small Molecular Antitumor Agent in Rat Models

After a week of acclimatization (about 200 g), the SD rats were divided into 10 rats per group. The hair on the back of the rats was shaved with an electric shaver on the day before the experiment, and then the experiment was performed. The small molecular antitumor agent was dissolved in a corresponding solvent for administration experiment, for which the types, administration dosage, mode and frequency were shown in Table 3. The antitumor agent was administered continually every day, until the rats developed rashes, at which the treatment experiment was performed. The experiment was divided into a delgocitinib group and a control group. During the treatment experiment, the administration experiment of the antitumor agents was continued. The back of the rats in the delgocitinib group (about 3 cm×3 cm) was applied with delgocitinib ointment once a day; the back of the rats in the control group (about 3 cm×3 cm) was applied with blank ointment. After applying the medicament, the rats were fixed by a cylinder for about 4 hrs. Then, the rats were released, wiped with clean water to remove the residual medicament at the application site, and returned to the cage. The experiments of administration of the antitumor agent and application of the ointment were repeated. The number of rats in the delgocitinib group on which the skin returned to normal or remarkably milder than the rashes of the rats in the control group at the end of experiment was calculated as the number of rats on which rashes had been effectively treated.

Table 3 lists various animal experiment combinations of small molecular antitumor agents and delgocitinib ointment, as well as the corresponding experimental results (wherein, the numerical values in the column of relative remission rate=the number of rats in the delgocitinib group on which rashes were milder than those of rats in the control group/the total number of rats in the delgocitinib group×100%).

TABLE 3
Experimental conditions and results of Examples 109-136

| | Small | | | | | | | | Relative |
|---|---|---|---|---|---|---|---|---|---|
| | molecular | | | | | Modeling | | | remission |
| Example | antitumor | | Dosage | Administration | | days for | Medicament | Concentration | rate |
| No. | agent | Target | (mg/kg) | mode | Frequency | Administration | applied | (wt %) | (%) |
| 109 | Afatinib | EGFR | 40 | Gavage | Once | 6 | Delgocitinib | 0.05% | 20.00% |
| | | | | | day | | ointment | | |
| 110 | Afatinib | EGFR | 40 | Gavage | Once a | 6 | Delgocitinib | 0.50% | 40.00% |
| | | | | | day | | ointment | | |
| 111 | Afatinib | EGFR | 40 | Gavage | Once a | 6 | Delgocitinib | 1.50% | 66.67% |
| | | | | | day | | ointment | | |
| 112 | Afatinib | EGFR | 40 | Gavage | Once a | 6 | Delgocitinib | 5.00% | 80.00% |
| | | | | | day | | ointment | | |
| 113 | Gefitinib | EGFR | 80 | Gavage | Twice a | 10 | Delgocitinib | 0.05% | 20.00% |
| | | | | | day | | ointment | | |
| 114 | Gefitinib | EGFR | 80 | Gavage | Twice a | 10 | Delgocitinib | 0.50% | 50.00% |
| | | | | | day | | ointment | | |
| 115 | Gefitinib | EGFR | 80 | Gavage | Twice a | 10 | Delgocitinib | 1.50% | 75.00% |
| | | | | | day | | ointment | | |
| 116 | Gefitinib | EGFR | 80 | Gavage | Twice a | 10 | Delgocitinib | 5.00% | 80.00% |
| | | | | | day | | ointment | | |
| 117 | Osimertinib | EGFR | 60 | Gavage | Twice a | 7 | Delgocitinib | 0.05% | 30.00% |
| | | | | | day | | ointment | | |
| 118 | Osimertinib | EGFR | 60 | Gavage | Twice a | 7 | Delgocitinib | 0.50% | 50.00% |
| | | | | | day | | ointment | | |
| 119 | Osimertinib | EGFR | 60 | Gavage | Twice a | 7 | Delgocitinib | 2.00% | 80.00% |
| | | | | | day | | ointment | | |
| 120 | Erlotinib | EGFR | 70 | Gavage | Once a | 10 | Delgocitinib | 0.50% | 30.00% |
| | | | | | day | | ointment | | |
| 121 | Erlotinib | EGFR | 70 | Gavage | Once a | 10 | Delgocitinib | 2.00% | 60.00% |
| | | | | | day | | ointment | | |
| 122 | Tirabrutinib | BTK | 40 | Gavage | Once a | 10 | Delgocitinib | 0.50% | 40.00% |
| | | | | | day | | ointment | | |
| 123 | Zanubrutinib | BTK | 25 | Gavage | Twice a | 10 | Delgocitinib | 1.50% | 80.00% |
| | | | | | day | | ointment | | |
| 124 | Acalabrutinib | BTK | 30 | Gavage | Once a | 10 | Delgocitinib | 0.50% | 30.00% |
| | | | | | day | | ointment | | |
| 125 | Binimetinib | MEK | 50 | Gavage | Once a | 10 | Delgocitinib | 2.00% | 60.00% |
| | | | | | day | | ointment | | |
| 126 | Cobimetinib | MEK | 25 | Gavage | Once a | 12 | Delgocitinib | 2.00% | 50.00% |
| | | | | | day | | ointment | | |
| 127 | Trametinib | MEK | 20 (iv) | Tail vein | 3 times | 10 | Delgocitinib | 2.00% | 60.00% |
| | | | | injection | a week | | ointment | | |
| 128 | Selumetinib | MEK | 100 | Gavage | Once a | 10 | Delgocitinib | 1.50% | 70.00% |
| | | | | | day | | ointment | | |
| 129 | Dactolisib | PI3K | 60 | Gavage | Once a | 13 | Delgocitinib | 2.00% | 60.00% |
| | | | | | day | | ointment | | |
| 130 | Apitolisib | PI3K | 30 | Gavage | Once a | 13 | Delgocitinib | 0.50% | 30.00% |
| | | | | | day | | ointment | | |
| 131 | Duvelisib | PI3K | 60 | Gavage | Twice a | 10 | Delgocitinib | 0.50% | 50.00% |
| | | | | | day | | ointment | | |
| 132 | Zotarolimus | mTORC1, | 4 (iv) | Tail vein | Once a | 14 | Delgocitinib | 0.05% | 20.00% |
| | | mTORC2 | | injection | day | | ointment | | |
| 133 | Sirolimus | mTORC1, | 15 | Gavage | Once a | 10 | Delgocitinib | 0.50% | 30.00% |
| | | mTORC2 | | | day | | ointment | | |
| 134 | Sorafenib | BRAF, | 60 | Gavage | Twice a | 10 | Delgocitinib | 0.05% | 20.00% |
| | | VEGFR | | | day | | ointment | | |
| 135 | Lenvatinib | VEGFR | 65 | Gavage | Once a | 12 | Delgocitinib | 0.50% | 30.00% |
| | | | | | day | | ointment | | |
| 136 | Regorafenib | BRAF, | 90 | Gavage | Once a | 12 | Delgocitinib | 2.00% | 60.00% |
| | | VEGFR | | | day | | ointment | | |

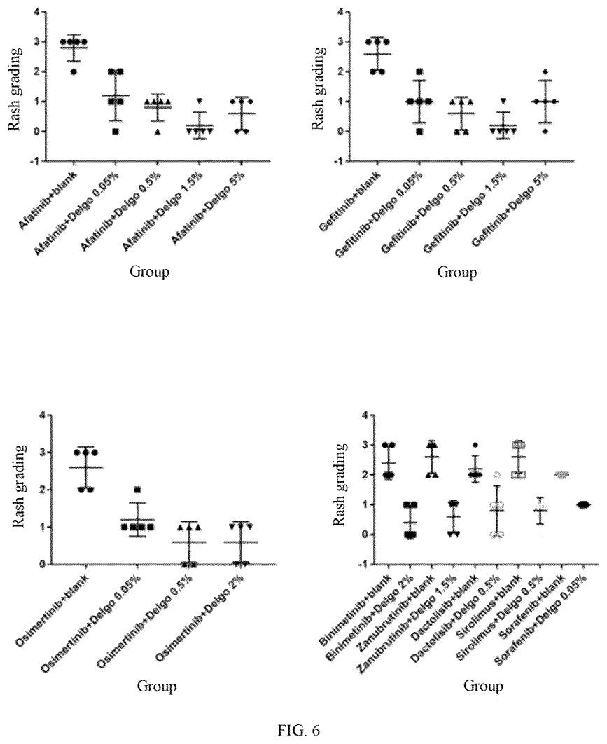

FIG. 5 shows the photographs of left side, back side, and right side of typical rats in the control group and the delgocitinib group in Table 3. FIG. 6 shows the rash grading of the delgocitinib group and the control group at the end of the experiment.

It can be seen from the results in Table 3 and FIGS. 5-6 that: the delgocitinib ointment can effectively treat rashes caused by a small molecular antitumor agent.

### Examples 137-144: Experiment for Demonstrating the Ability of Delgocitinib in Treating Rashes Caused by an Antibody-Type Antitumor Agent in Rat Models

After a week of acclimatization (about 200 g), the SD rats were divided into 10 rats per group. The hair on the back of the rats was shaved with an electric shaver on the day before the experiment, and then the administration experiment was performed. The experiment was divided into a delgocitinib group and a control group. The administration frequency, dosage and injection rate of the antibody-type antitumor agents were shown in Table 4. The administration experiment of antibody-type antitumor agents was continued until the rats developed rashes, at which the treatment experiment was performed. The experiment was divided into a delgocitinib group and a control group. During the treatment experiment, the administration experiment of the antibody-type antitumor agents was continued. The back of the rats in the delgocitinib group (about 3 cm×3 cm) was applied with delgocitinib ointment once a day; the back of the rats in the control group (about 3 cm×3 cm) was applied with blank ointment. After applying the medicament, the rats were fixed by a cylinder for about 4 hrs. Then, the rats were released, wiped with clean water to remove the residual medicament at the application site, and returned to the cage. The experiments of administration of the antibody-type antitumor agents and application of the ointment were repeated. The number of rats in the delgocitinib group on which the skin returned to normal or milder than the rashes of the rats in the control group at the end of experiment was calculated as the number of rats on which rashes had been effectively treated.

Table 4 lists various animal experiment combinations of antibody-type antitumor agents and delgocitinib ointment, as well as the corresponding experimental results (wherein, the numerical values in the column of relative remission rate=the number of rats in the delgocitinib group on which rashes were milder than those of rats in the control group/the total number of rats in the delgocitinib group×1000%).

TABLE 4
Experimental conditions and results of Examples 137-144

| | Antibody- | | | | | | | | Relative |
|---|---|---|---|---|---|---|---|---|---|
| | type | | | | | | | | remission |
| Example | antitumor | | | Administration | | Modeling | Medicament | Concentration | rate |
| No. | agent | Target | Dosage | mode | Frequency | days | applied | wt % | (%) |
| 137 | Cetuximab | EGFR | 100 mg/kg, 15 | Tail vein | Twice a | 16 | Delgocitinib | 0.05% | 20% |
| | | | min | injection | week | | ointment | | |
| | | | with an | | | | | | |
| | | | injection rate | | | | | | |
| | | | of 1.3 | | | | | | |
| | | | ml/kg/min for | | | | | | |
| | | | each time | | | | | | |
| 138 | Cetuximab | EGFR | 100 mg/kg, 15 | Tail vein | Twice a | 16 | Delgocitinib | 0.50% | 40% |
| | | | min | injection | week | | ointment | | |
| | | | with an | | | | | | |
| | | | injection rate | | | | | | |
| | | | of 1.3 | | | | | | |
| | | | ml/kg/min for | | | | | | |
| | | | each time | | | | | | |
| 139 | Cetuximab | EGFR | 100 mg/kg, 15 | Tail vein | Twice a | 16 | Delgocitinib | 1.50% | 80% |
| | | | min | injection | week | | ointment | | |
| | | | with an | | | | | | |
| | | | injection rate | | | | | | |
| | | | of 1.3 | | | | | | |
| | | | ml/kg/min for | | | | | | |
| | | | each time | | | | | | |
| 140 | Cetuximab | EGFR | 100 mg/kg, 15 | Tail vein | Twice a | 16 | Delgocitinib | 2.00% | 70% |
| | | | min | injection | week | | ointment | | |
| | | | with an | | | | | | |
| | | | injection rate | | | | | | |
| | | | of 1.3 | | | | | | |
| | | | ml/kg/min for | | | | | | |
| | | | each time | | | | | | |
| 141 | Panitumumab | EGFR | 60 (iv) | Tail vein | Once a | 14 | Delgocitinib | 0.05% | 30% |
| | | | | injection | week | | ointment | | |
| | | | with an | | | 14 | Delgocitinib | 1.50% | 60% |
| | | | injection rate | | | | ointment | | |
| | | | of 1.3 | | | | | | |
| | | | ml/kg/min for | | | | | | |
| | | | each time | | | | | | |
| 142 | Rituximab | CD20 | 60 (iv) | Tail vein | Once a | 18 | Delgocitinib | 0.50% | 60% |
| | | | | injection | week | | ointment | | |
| | | | with an | | | 18 | Delgocitinib | 1.50% | 70% |
| | | | injection rate | | | | ointment | | |
| | | | of 1.3 | | | | | | |
| | | | ml/kg/min for | | | | | | |
| | | | each time | | | | | | |
| 143 | Amivantamab | EGFR/ | 30 | Tail vein | Twice a | 10 | Delgocitinib | 0.05% | 40% |
| | | c-Met | with an | injection | week | | ointment | | |
| | | | injection rate | | | | | | |
| | | | of 1.3 | | | | | | |
| | | | ml/kg/min for | | | | | | |
| | | | each time | | | | | | |
| 144 | Amivantamab | EGFR/ | 30 | Tail vein | Twice a | 10 | Delgocitinib | 1.5% | 70% |
| | | c-Met | with an | injection | week | | ointment | | |
| | | | injection rate | | | | | | |
| | | | of 1.3 | | | | | | |
| | | | ml/kg/min for | | | | | | |
| | | | each time | | | | | | |

FIG. 7 shows the rash grading of the delgocitinib group and the control group at the end of the experiment.

It can be seen from the results in Table 4 and FIG. 7 that: Delgocitinib ointment can effectively treat rashes caused by antibody-type antitumor agents.

### Examples 145-157: Comparison of the Delgocitinib Ointment with Other Currently Clinically Available Dermatological Medicaments in the Experiment of Preventing Rashes Caused by Small Molecular Antitumor Agents

After a week of acclimatization (about 200 g), the SD rats were divided into 10 rats per group. The hair on the back of the rats was shaved with an electric shaver on the day before the experiment, and then the administration experiment was performed. The small molecular antitumor agent was dissolved in a suitable solvent for gavage/injection administration to all the rats, for which the administration mode, dosage and frequency were shown in Table 5. The experiment was divided into a delgocitinib group and a clinical medication group. After administration of the antitumor agent to rats, the back of the rats in the delgocitinib group (about 3 cm×3 cm) was applied with delgocitinib ointment (the concentration thereof was shown in Table 5, about 0.5 g); the back of the rats in the clinical medication group (about 3 cm×3 cm) was applied with currently clinically available dermatological medicaments (Examples 145-157). After applying the medicament, the rats were fixed by a cylinder for about 4 hrs. Then, the rats were released, wiped with clean water to remove the residual medicament at the application site, and returned to the cage for normal breeding. Delgocitinib and the clinical medication ointment were applied once a day. The experiments of administration of the antitumor agent and application of the ointment were repeated until the rats in the clinical medication group developed apparent rashes. At this point, the number of rats in the delgocitinib group on which the skin kept normal or remarkably milder than the rashes of the rats in the clinical medication group was calculated as the number of rats on which rashes had been effectively inhibited.

Table 5 lists various animal experiment combinations of delgocitinib ointment and currently clinically available dermatological medicaments, as well as the corresponding experimental results (wherein, the numerical values in the column of relative remission rate=the number of rats in the delgocitinib group on which rashes were milder than those of rats in the clinical medication group/the total number of rats in the delgocitinib group×100%).

TABLE 5
Experimental conditions and results of Examples 145-157

| | | | | | | Medicament applied | | | |
|---|---|---|---|---|---|---|---|---|---|
| | Small | | | | | | Other | Days | |
| | molecular | | | | | Delgocitinib | dermatologic | for | Relative |
| Example | antitumor | | | Administration | | ointment | medicament | medicament | remission |
| No. | agent | Target | Dosage | mode | Frequency | group | group | application | rate |
| 145 | Gefitinib | EGFR | 80 | Gavage | Twice a day | 0.05% | Hydrocortisone | 15 | 40% |
| | | | mg/kg | | | | ointment | | |
| | | | | | | | (1%) | | |
| 146 | Gefitinib | EGFR | 80 | Gavage | Twice a day | 0.50% | Hydrocortisone | 15 | 80% |
| | | | mg/kg | | | | ointment | | |
| | | | | | | | (1%) | | |
| 147 | Gefitinib | EGFR | 80 | Gavage | Twice a day | 1.50% | Hydrocortisone | 15 | 100% |
| | | | mg/kg | | | | ointment | | |
| | | | | | | | (1%) | | |
| 148 | Gefitinib | EGFR | 80 | Gavage | Twice a day | 5% | Hydrocortisone | 15 | 100% |
| | | | mg/kg | | | | ointment | | |
| | | | | | | | (1%) | | |
| 149 | Afatinib | EGFR | 40 | Gavage | Once a day | 0.50% | Hydrocortisone | 14 | 60% |
| | | | mg/kg | | | | ointment | | |
| | | | | | | | (1%) | | |
| 150 | Osimertinib | EGFR | 60 | Gavage | Twice a day | 1.50% | Hydrocortisone | 10 | 80.00% |
| | | | mg/kg | | | | ointment | | |
| | | | | | | | (1%) | | |
| 151 | EAI045 | EGFR | 80 | Gavage | Once a day | 5% | Hydrocortisone | 11 | 100% |
| | | | mg/kg | | | | ointment | | |
| | | | | | | | (1%) | | |
| 152 | Afatinib | EGFR | 40 | Gavage | Once a day | 1.50% | Desonide | 14 | 100% |
| | | | mg/kg | | | | ointment | | |
| | | | | | | | (0.05%) | | |
| 153 | Afatinib | EGFR | 40 | Gavage | Once a day | 1.50% | Clindamycin | 14 | 100% |
| | | | mg/kg | | | | ointment (1%) | | |
| 154 | Dacomitinib | EGFR | 10 | Gavage | Once a day | 1.00% | Fluocinonide | 12 | 80% |
| | | | mg/kg | | | | ointment | | |
| | | | | | | | (0.05%) | | |
| 155 | Dactolisib | PI3K | 60 | Gavage | Once a day | 2.00% | Alclometasone | 12 | 100% |
| | | | mg/kg | | | | ointment | | |
| | | | | | | | (0.05%) | | |
| 156 | Selumetinib | MEK | 100 | Gavage | Once a day | 1.00% | Desonide | 12 | 80% |
| | | | mg/kg | | | | ointment | | |
| | | | | | | | (0.05%) | | |
| 157 | Sorafenib | BRAF, | 60 | Gavage | Twice a day | 2.00% | Desonide | 14 | 100% |
| | | VEGFR | mg/kg | | | | ointment | | |
| | | | | | | | (0.05%) | | |

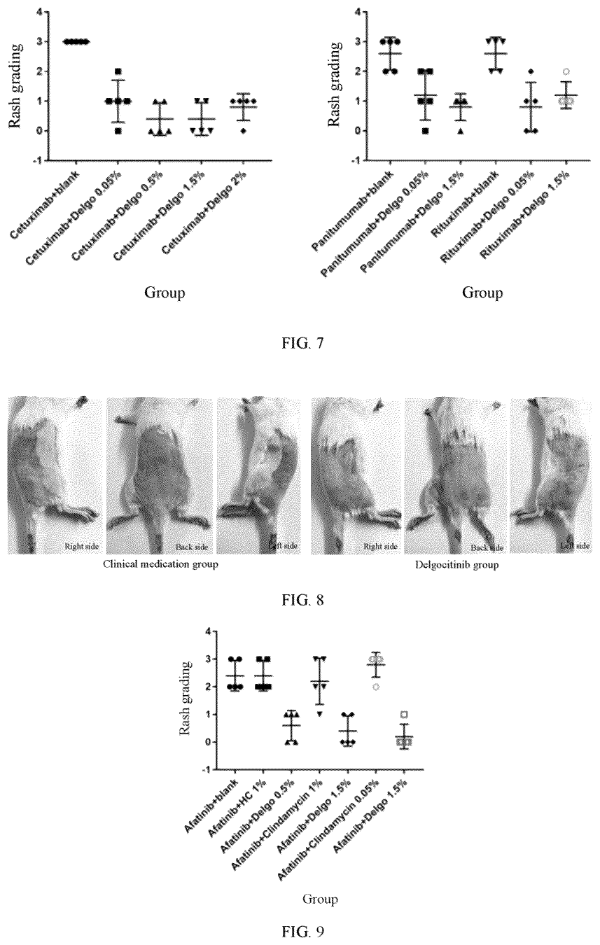

FIG. 8 shows the photographs of left side, back side, and right side of typical rats in the clinical medication group and the delgocitinib group in Table 5. FIG. 9 shows the rash grading of the delgocitinib group and the clinical medication group at the end of the experiment.

It can be seen from the results in Table 5 and FIG. 9 that: compared with the currently clinically available dermatological medicaments (almost having no therapeutic effects on rashes caused by a small molecular antitumor agent), delgocitinib ointment can effectively control rashes caused by a small molecular antitumor agent.

### Examples 158-159: Comparison of the Delgocitinib Ointment with Other Currently Clinically Available Dermatological Medicaments in the Experiment of Preventing Rashes Caused by Antibody-Type Antitumor Agents

After a week of acclimatization (about 200 g), the SD rats were divided into 10 rats per group. The hair on the back of the rats was shaved with an electric shaver on the day before the experiment, and then the administration experiment was performed. The experiment was divided into a delgocitinib group and a clinical medication group. The administration frequency, dosage and injection rate of the antibody-type antitumor agents were shown in Table 6. During the experiment, the back of the rats in the delgocitinib group (about 3 cm×3 cm) was applied with delgocitinib ointment; the back of the rats in the clinical medication group (about 3 cm×3 cm) was applied with a currently clinically available dermatological medicament. After applying the medicament, the rats were fixed by a cylinder for about 4 hrs. Then, the rats were released, wiped with clean water to remove the residual medicament at the application site, and returned to the cage for normal breeding. The ointment was applied once a day. The experiment ended when the rats in the clinical medication group developed apparent rashes. The number of rats in the delgocitinib group on which the skin kept normal or remarkably milder than the rashes of the rats in the clinical medication group at the end of the experiment was calculated as the number of rats on which rashes had been effectively inhibited.

Table 6 lists animal experiment combinations of delgocitinib ointment and currently clinically available dermatological medicaments, as well as the corresponding experimental results (wherein, the numerical values in the column of relative remission rate=the number of rats in the delgocitinib group on which rashes were milder than those of rats in the clinical medication group/the total number of rats in the delgocitinib group×100%).

TABLE 6
Experimental conditions and results of examples 158-159

| | | | | | | Medicament applied | | | |
|---|---|---|---|---|---|---|---|---|---|
| | Antibody- | | | | | | Other | | |
| | type | | | | | Delgocitinib | dermatologic | Days for | Relative |
| Example | antitumor | | | Administration | | ointment | medicament | medicament | remission |
| No. | agent | Target | Dosage | mode | Frequency | group | group | application | rate |
| 158 | Cetuximab | EGFR | 100 | Tail vein | Twice | 2.00% | Desonide | 10 | 70% |
| | | | mg/kg, 15 | injection | week | | ointment | | |
| | | | min | | | | (0.05%) | | |
| 159 | | | with an | | | 1.50% | Hydrocortisone | 10 | 60% |
| | | | injection | | | | ointment | | |
| | | | rate of 1.3 | | | | (1%) | | |
| | | | ml/kg/min | | | | | | |
| | | | for each | | | | | | |
| | | | time | | | | | | |

It can be seen from the results in Table 6 that: compared with the currently clinically available dermatological medicaments (almost having no therapeutic effects on rashes caused by an antibody-type antitumor agent), delgocitinib ointment can effectively control rashes caused by an antibody-type antitumor agent.

### Examples 160-169: Clinical Effect of Delgocitinib on Rashes Caused by an Antitumor Agent

The test subjects come from patients who have received target therapy and/or immunotherapy and have developed rashes. The patients receiving target therapy are being treated with cetuximab, or other antibody-type antitumor agents; the patients receiving immunotherapy are being treated with CTLA-4 inhibitor (e.g.: Ipilimumab) and/or PD-1/PD-L1 inhibitor (e.g.: Pembrolizumab, Nivolumab, etc.). For patients who meet the diagnostic criteria for rash and had been evaluated as Grade 1 or above in accordance with NCI-CTCAE v5.0, the symptoms last more than a week.

The diagnostic criteria for rash can refer to NCI-CTCAE v5.0 and ASCO Guideline, by which the rashes caused by targeted therapy and immunotherapy are classified separately, in particular:

Rash caused by targeted therapy:
Grade 1: erythema, papules, vesicle and/or pustules, covering<10% of body surface area, which may or may not be associated with symptoms of pruritus, burning, tenderness or tight skin;
Grade 2: erythema, papules, vesicle and/or pustules, covering 10%-30% of body surface area, which may or may not be associated with symptoms of pruritus, burning, tenderness or tight skin, associated with psychosocial impact, limiting instrumental activities of daily living (ADL); rash covering>30% of body surface area, with or without mild symptoms;
Grade 3: erythema, papule, vesicle and/or pustule, covering>30% of body surface area, with moderate or severe symptoms; limited self-care ADL;
Grade 4: life-threatening consequences; papule and/or pustule covering any percent of body surface area, which may or may not be associated with symptoms of pruritus or tenderness;
Grade 5: Death.

Rashes caused by immunotherapy:
Grade 1: rash covering<10% of body surface area, which may or may not be associated with symptoms;
Grade 2: rash covering 10%-30% of body surface area, which may or may not be associated with symptoms, limiting instrumental ADL;
Grade 3: rash covering>30% of body surface area, which may or may not be associated with symptoms, limited self-care ADL;
Grade 4: rash covering>30% of body surface area, with infection or other complication, in need of hospitalization.

The experiment was divided into a treatment group and a control group. In the course of targeted therapy and immunotherapy, for the treatment group: the area of rash was locally rinsed with clean water, and the affected part was applied with the delgocitinib ointment (Ointment I, an ointment on the market) 3 times a day: in the morning, at noon, and in the evening; for the control group: the area of rash was locally rinsed with clean water, and the affected part was applied with a blank ointment (Ointment II) 3 times a day: in the morning, at noon, and in the evening; with 4 weeks as a course of treatment. Patients were followed up weekly by telephone, and a clinical evaluation form was filled in. The evaluation form consists of 9 items: prior treatment, treatment containing ointment I/ointment II, home care, auxiliary treatment, wound types, lesion assessment (with the width and length measured in centimeters), skin surrounding the lesion, assessment of life quality, and assessment of whether to suspend medication. If necessary, skin biopsy should be performed, and evaluated by pathologists.

The number, size and changing trend of rashes were evaluated weekly at the application site and non-application site. The therapeutic effect was evaluated by a method as below:
Clinically controlled: symptoms disappeared at the end of the treatment;
Significant effective: the grading of symptoms was reduced by 2 grades at the end of the treatment;
Effective: the grading of symptoms was reduced by 1 grade at the end of the treatment;
Ineffective: patients failing to meet the above criteria.

The above evaluation method of therapeutic effect was used to calculate the remission rate of rashes (Clinically controlled+Significant effective+Effective)/Total cases in this group*100%.

Table 7 lists different combinations of antitumor agents and ointment, where the relative remission rate=(Clinically controlled+Significant effective+Effective)/Total cases in this group*100%.

TABLE 7
Experimental conditions and results of Examples 160-169

| | | | | | Relative |
|---|---|---|---|---|---|
| Example | Antitumor | | Medicament | | remission |
| No. | agent | Target | applied | Frequency | rate |
| 160 | Cetuximab | EGFR | 0.5% Delgocitinib | 3 times a day | 60% |
| | | | ointment | | |
| 161 | | | Blank ointment | 3 times a day | 10% |
| 162 | Panitumumab | EGFR | 0.5% Delgocitinib | 3 times a day | 70% |
| | | | ointment | | |
| 163 | | | Blank ointment | 3 times a day | 20% |
| 164 | Ipilimumab | CTLA-4 | 0.5% Delgocitinib | 3 times a day | 50% |
| | | | ointment | | |
| 165 | | | Blank ointment | 3 times a day | 20% |
| 166 | Pembrolizumab | PD-1 | 0.5% Delgocitinib | 3 times a day | 60% |
| | | | ointment | | |
| 167 | | | Blank ointment | 3 times a day | 10% |
| 168 | Ipilimumab + | PD-1 + | 0.5% Delgocitinib | 3 times a day | 40% |
| | Nivolumab | CTLA-4 | ointment | | |
| 169 | | | Blank ointment | 3 times a day | 10% |

It can be seen from the results in Table 7 that: Delgocitinib ointment has a certain relieving effect on rashes developed on patients who had received a targeted therapy (Cetuximab, Panitumumab) and an immunotherapy (CTLA-4 inhibitor, and/or PD-1/PD-L1 inhibitor).

### Examples 170-178: Experiment for Demonstrating the Ability of Delgocitinib in Preventing the Skin Disease or Disorder Associated with Antitumor Agent in Rat Models

Construction of rat rash models: An antitumor agent was daily administered to 6-week female SD rats, and after several days, a large area of rashes appeared on the back of the rats. There was no difference between the left and right sides of the rash area, and the rash grade was similar on both sides, which were like the conditions in human. Thus, rats are very good animal models to mimic the rashes caused by the antitumor agent.

After a week of acclimatization (about 200 g), the SD rats were divided into 10 rats per group. The hair on the back of the rats was shaved with an electric shaver on the day before the experiment, and then the administration experiment was performed. The antitumor agents were dissolved in sterile water and diluted with a PBS buffer solution. The dose was no more than 2 mL per gavage, and the dosages were shown in Table 8. The experiment was divided into a delgocitinib group and a control group. After gavage, the back of the rats in the delgocitinib group (about 3 cm×3 cm) was applied with delgocitinib ointment (the type and concentration were shown in Table 8); the back of the rats in the control group (about 3 cm×3 cm) was applied with blank ointment (about 0.5 g). After applying the medicament, the rats were fixed by a cylinder for about 4 hrs. Then, the rats were released, wiped with clean water to remove the residual medicament at the application site, and returned to the cage for normal breeding. The gavage frequency of the antitumor agents as shown in the table below, but the delgocitinib and the blank ointment were applied once a day. The experiments of administration of the antitumor agent and application of the ointment were repeated until the rats in the control group developed apparent rashes. At this point, the number of rats in the delgocitinib group on which the skin kept normal or remarkably milder than the rashes of the rats in the control group was calculated as the number of rats on which rashes had been effectively inhibited.

Table 8 lists various animal experiment combinations of antitumor agents and delgocitinib ointment, as well as the corresponding experimental results (wherein, the numerical values in the column of control rate=the number of rats in the delgocitinib group on which rashes were milder than those of rats in the control group/the total number of rats in the delgocitinib group×100%).

TABLE 8
Experimental conditions and results of Examples 170-178

| Examples | | Type of the | Dosage | | | Concentration | | Control |
|---|---|---|---|---|---|---|---|---|
| No. | EGFR inhibitor | inhibitor | (mg/kg) | Frequency | Administration | wt % | Days | rate |
| 170 | Lazertinib | Small | 80 | Once a day | Delgocitinib | 0.5% | 16 | 40% |
| | | molecule | | | ointment | | | |
| | | EGFR | | | | | | |
| | | inhibitor | | | | | | |
| 171 | Lazertinib | Small | 80 | Once a day | Delgocitinib | 2.0% | 16 | 60% |
| | | molecule | | | ointment | | | |
| | | EGFR | | | | | | |
| | | inhibitor | | | | | | |
| 172 | Alflutinib | Small | 50 | Once a day | Delgocitinib | 1.0% | 14 | 50% |
| | | molecule | | | ointment | | | |
| | | EGFR | | | | | | |
| | | inhibitor | | | | | | |
| 173 | Alflutinib | Small | 50 | Once a day | Delgocitinib | 5.0% | 14 | 80% |
| | | molecule | | | ointment | | | |
| | | EGFR | | | | | | |
| | | inhibitor | | | | | | |
| 174 | Olmutinib | Small | 100 | Once a day | Delgocitinib | 0.5% | 18 | 30% |
| | | molecule | | | ointment | | | |
| | | EGFR | | | | | | |
| | | inhibitor | | | | | | |
| 175 | Olmutinib | Small | 100 | Once a day | Delgocitinib | 2.0% | 18 | 90% |
| | | molecule | | | ointment | | | |
| | | EGFR | | | | | | |
| | | inhibitor | | | | | | |
| 176 | Olmutinib | Small | 100 | Once a day | Delgocitinib | 5.0% | 18 | 90% |
| | | molecule | | | ointment | | | |
| | | EGFR | | | | | | |
| | | inhibitor | | | | | | |
| 177 | Icotinib | Small | 150 | Twice a | Delgocitinib | 1.0% | 12 | 60% |
| | | molecule | | day | ointment | | | |
| | | EGFR | | | | | | |
| | | inhibitor | | | | | | |
| 178 | Icotinib | Small | 150 | Twice a | Delgocitinib | 3.0% | 12 | 100% |
| | | molecule | | day | ointment | | | |
| | | EGFR | | | | | | |
| | | inhibitor | | | | | | |

### Examples 179: Experiment for Demonstrating the Ability of Delgocitinib in Treating Skin Disease or Disorder Associated with Antitumor Agent in Rat Models

After a week of acclimatization (about 200 g), the SD rats were divided into 10 rats per group. The hair on the back of the rats was shaved with an electric shaver on the day before the experiment, and then the experiment was performed. The antitumor agents were dissolved in sterile water and diluted with a PBS buffer solution. The dose was no more than 2 mL per gavage, and the dosages were shown in Table 9. The antitumor agent was administered continually every day, until the rats developed rashes, at which the treatment experiment was performed. The experiment was divided into a delgocitinib group and a control group. During the treatment experiment, the administration experiment of the antitumor agents was continued. After gavage, the back of the rats in the delgocitinib group (about 3 cm×3 cm) was applied with delgocitinib ointment once a day; the back of the rats in the control group (about 3 cm×3 cm) was applied with blank ointment. After applying the medicament, the rats were fixed by a cylinder for about 4 hrs. Then, the rats were released, wiped with clean water to remove the residual medicament at the application site, and returned to the cage. The gavage frequency of the antitumor agents as shown in the Table 9, but the delgocitinib and the blank ointment were applied once a day. The experiments of administration of the antitumor agent and application of the ointment were repeated. The number of rats in the delgocitinib group on which the skin returned to normal or remarkably milder than the rashes of the rats in the control group at the end of experiment was calculated as the number of rats on which rashes had been effectively treated.

Table 9 lists animal experiment combination of small molecular antitumor agent and delgocitinib ointment, as well as the corresponding experimental result (wherein, the numerical value in the column of control rate=the number of rats in the delgocitinib group on which rashes were milder than those of rats in the control group/the total number of rats in the delgocitinib group×100%).

TABLE 9
Experimental conditions and results of Examples 179

| | | Type of | | | | | | |
|---|---|---|---|---|---|---|---|---|
| | | the | | | Modeling | | Concentration | Control |
| Examples | Inhibitor | inhibitor | Dosage | Frequency | days | Administration | wt % | Rate |
| 179 | GSK- | Small | 10 | Once a | 10 | Delgocitinib | 0.2% | 80% |
| | 1120212 | molecule | | day | | ointment | | |
| | | MEK | | | | | | |
| | | inhibitor | | | | | | |

The foregoing detailed description is provided by means of explanation and examples, but is not intended to limit the scope of the attached claims. Various changes to the embodiments listed in the present application currently are apparent to those with ordinary skills in the art, and are retained in the attached claims and their equivalent scope.

1-86. (canceled)

87: A method for preventing, relieving and/or treating a skin disease or disorder associated with an antitumor agent, comprising administrating a compound as shown in Formula I or a pharmaceutically acceptable salt, prodrug, isotopic variant or solvate thereof to a subject in need thereof, 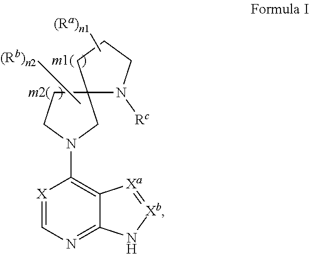
wherein:
R^{a} is the same or different and each is: (1) C₁₋₆ alkyl, or (2) halogen atom,
n1 is an integer selected from 0 to 4,
R^{b} is the same or different and each is: (1) C₁₋₆ alkyl, or (2) halogen atom,
n2 is an integer selected from 0 to 4,
m1 is an integer selected from 0 to 3,
m2 is an integer selected from 1 to 4,
X^{a}═X^{b} is (1) CH═CH, (2) N═CH, or (3) CH═N,
X is (1) nitrogen atom, or (2) C—R^{d}, wherein R^{d} is hydrogen atom or halogen atom,
R^{c} is a group selected from the following (1) to (6):
(1) hydrogen atom,
(2) C₁₋₆ alkyl optionally substituted by the same or different 1 to 5 substituents selected from the following Group A,
(3) —C(═O)—R^{c1},
(4) —C(═O)—O—R^{c2},
(5) —C(═O)—NR^{c3}R^{c4}, wherein R^{c1}, R^{c2}, R^{c3} and R^{c4} are the same or different and each is: (i) hydrogen atom, or (ii) C₁₋₆ alkyl optionally substituted by the same or different 1 to 5 substituents selected from the following Group A, or
(6) a group with a structure of 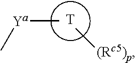
wherein, Y^{a} is a group selected from the following (i) to (iii): (i) C₁₋₆ alkylene, (ii) —C(═O)—, or (iii) —C(═O)—O—,
Ring T is: (i) C₆₋₁₀ aryl, (ii) C₃₋₁₀ cycloalkyl, or (iii) saturated monoheterocyclyl containing 1 to 4 heteroatoms selected from nitrogen atom, oxygen atom or sulfur atom as well as carbon atoms, and the number of the constituent ring atoms is 3 to 7,
R^{c5} is the same or different and each is: (i) cyanogroup, or (ii) nitro, p is an integer selected from 0 to 4,
Group A is the group consisting of: (a) hydroxyl, (b) C₁₋₆ alkoxy, (c) cyanogroup, (d) C₁₋₆ alkoxycarbonyl, (e) C₁₋₆ alkylcarbonyloxy, and (f) C₂₋₆ alkenyloxy.

88: The method of claim 87, wherein m1 is 0, m2 is 1, the compound is a compound of Formula IV: 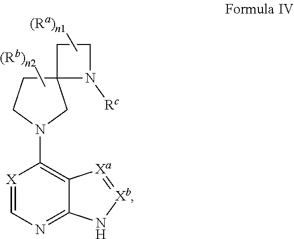
wherein R^{a}, R^{b}, R^{c}, X, X^{a}, X^{b}, n1 and n2 have the same meaning as defined in claim 87.

89: The method of claim 87, wherein X^{a}═X^{b} is CH═CH, and X is a nitrogen atom.

90: The method of claim 87, wherein n1 is 1, and n2 is 0.

91: The method of claim 87, wherein R^{a} is a methyl or a fluorine atom.

92: The method of claim 87, wherein R^{c} is —C(═O)—R^{c3}R^{c4}, R^{c3} is C₁₋₆ alkyl substituted by one cyanogroup, and R^{c4} is a hydrogen.

93: The method of claim 87, wherein the compound is

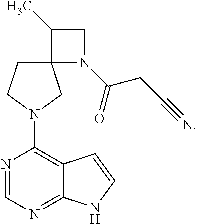 [SMILES: CC1CN(C(=O)CC#N)C12CCN(c1ncnc3[nH]ccc13)C2]

94: The method of claim 87, wherein the antitumor agent comprises a small molecular compound, a small molecular conjugate, protein and/or a polynucleotide.

95: The method of claim 87, wherein the antitumor agent comprises a targeted therapeutic agent and/or an immunotherapeutic agent.

96: The method of claim 95, wherein the targeted therapeutic agent comprises a small molecular compound and/or an antibody or fragment of an antigen binding thereof.

97: The method of claim 95, wherein the targeted therapeutic agent targets a tumor antigen.

98: The method of claim 95, wherein the targeted therapeutic agent targets EGFR, ALK, MEK, VEGFR, FGFR, PDGFR, ABL, BTK, KIT, AKT, TORC, HER2, HER3, HER4, PI3K, CDK, JAK, ROS1, RET, MET, KRAS, BRAF, BCRP, NTRK, RAS, MSI, PR/ER, BCR/ABL, HDAC, FAK, PYK2, CD20, PD-L1 and/or BRCA1/2, or mutants thereof.

99: The method of claim 95, wherein the targeted therapeutic agent is EGFR inhibitor, MEK inhibitor, ALK inhibitor, BTK inhibitor, PI3K inhibitor, AKT inhibitor, VEGFR inhibitor, mTOR inhibitor, HDAC inhibitor, KIT inhibitor, FGFR inhibitor, FAK inhibitor, BCRP inhibitor, EGFR/cMET inhibitor and/or SRC inhibitor, as well as a combination thereof.

100: The method of claim 95, wherein the immunotherapeutic agent is an immune checkpoint inhibitor, a modified immune cell and/or a vaccine.

101: The method of claim 95, wherein the immunotherapeutic agent is a PD-1 inhibitor, a PD-L1 inhibitor and/or a CTLA-4 inhibitor.

102: The method of claim 87, wherein the skin disease or disorder comprises a cutaneous disease and/or a subcutaneous tissue disease caused by the antitumor agent.

103: The method of claim 87, wherein the skin disease or disorder is a rash.

104: The method of claim 87, wherein the subject comprises a cancer patient.

105: The method of claim 87, wherein the compound is prepared for transdermal administration.

106: The method of claim 87, wherein the dosage of the compound as shown in Formula I or a pharmaceutically acceptable salt, prodrug, isotopic variant or solvate thereof is about 0.01% to about 5.0%.
